(19) United States
(12) Patent Application Publication
(10) Pub. No.: US 2026/0351338 A1
(43) Pub. Date: Nov. 5, 2026

(54) STEERED MOLECULAR DYNAMICS BASED NATIVE POSE AND AFFINITY PREDICTION FOR PROTEIN-LIGAND COMPLEXES FORMED THROUGH CONSTRAINED DOCKING

(71) Applicant: Genentech, Inc., South San Francisco, CA (US)

(72) Inventors: Franziska SEEGER, Seattle, WA (US); Andrew Martin WATKINS, San Francisco, CA (US); Robert Gregory ALBERSTEIN, South San Francisco, CA (US); Darcy S. DAVIDSON, South San Francisco, CA (US)

(21) Appl. No.: 19/708,276

(22) Filed: Jun. 15, 2026

Related U.S. Application Data
(63) Continuation of application No. PCT/US2024/060036, filed on Dec. 13, 2024.
(60) Provisional application No. 63/610,967, filed on Dec. 15, 2023.

(51) Int. Cl. G16B 15/30; G06F 30/20; G16B 20/30

(52) U.S. Cl. CPC G16B 15/30; G06F 30/20; G16B 20/30

(57) ABSTRACT

A binding site in a ligand forming an interface between the ligand and a protein molecule may be identified. Protein-ligand complexes may be generated by sampling different poses of the ligand in a binding site of the protein molecule with the different poses being constrained by the binding site of the ligand. Multiple rounds of steered molecular dynamics (SMD) simulation may be performed for each protein-ligand complex to determine a binding free energy of each protein-ligand complex. The binding affinity between the ligand and the protein molecule may be determined based on the binding free energy of each protein-ligand complex. A protein-ligand complex corresponding to the native pose of the protein molecule bound to the ligand may also be identified based on the binding free energy of each protein-ligand molecule.

### CROSS REFERENCE OT RELATED APPLICATION

This application claims priority to U.S. Provisional Application No. 63/610,967, entitled “STEERED MOLECULAR DYNAMICS BASED NATIVE POSE AND AFFINITY PREDICTION FOR PROTEIN-LIGAND COMPLEXES FORMED THROUGH CONSTRAINED DOCKING” and filed on Dec. 15, 2023, the disclosure of which is incorporated herein by reference in its entirety.

### TECHNICAL FIELD

The subject matter described herein relates generally to molecular dynamics simulations and more specifically to steered molecular dynamics (SMD) simulations for predicting the native pose of a ligand bound to a protein molecule and the binding affinity therebetween.

### BACKGROUND

Molecular binding is a process in which two or more molecules (e.g., proteins, ligands, etc.) interact to form a molecular structure, such as a protein-ligand complex. For example, molecular binding can include intermolecular interactions (e.g., electrostatics, van der Waals forces, and/or the like) between a first molecule and a second molecule. The portion of the first molecule that participates in the chemical bonding with the second molecule can include one or more binding sites. For instance, in the case of a protein-ligand complex formed by the binding interaction between a protein molecule and a ligand, each binding site in the protein molecule may include one or more amino acid residues that participate in the binding interaction with the ligand. Where the ligand is another protein molecule, the binding sites in the ligand may also include one or more of the amino acid residues that participate in the binding interaction. In instances where the ligand is a small molecule, such as a lipid, a sugar, or a small molecule drug, the binding sites in the ligand may include one or more of the atoms that participate in the binding interaction with the protein molecule. Where the ligand is a nucleic acid, the binding sites in the ligand may include one or more of the nucleotides forming the nucleic acid.

### SUMMARY

Systems, methods, and articles of manufacture, including computer program products, are provided for determining the binding affinity between a ligand and a protein molecule by at least performing one or more rounds of steered molecular dynamics (SMD) simulation to dissociate protein-ligand complexes formed through constrained docking. In one aspect, there is provided a system including at least one data processor and at least one memory. The at least one memory may store instructions, which when executed by the at least one data processor, result in operations comprising: identifying at least a portion of a binding site in a ligand forming an interface between the ligand and a protein molecule; generating one or more protein-ligand complexes by at least sampling a plurality of different poses of the ligand in a binding site in the protein molecule, the plurality of different poses being constrained by the binding site of the ligand such that each pose of the plurality of poses orients the binding site of the ligand to bind to the binding site of the protein molecule; performing, for each protein-ligand complex of the one or more protein-ligand complexes, a steered molecular dynamics (SMD) simulation to determine a binding free energy of each protein-ligand complex; and determining, based at least on the binding free energy of each protein-ligand complex, a binding affinity between the ligand and the protein molecule.

In another aspect, there is provided a method for determining the binding affinity between a ligand and a protein molecule. The method may include: identifying at least a portion of a binding site in a ligand forming an interface between the ligand and a protein molecule; generating one or more protein-ligand complexes by at least sampling a plurality of different poses of the ligand in a binding site in the protein molecule, the plurality of different poses being constrained by the binding site of the ligand such that each pose of the plurality of poses orients the binding site of the ligand to bind to the binding site of the protein molecule; performing, for each protein-ligand complex of the one or more protein-ligand complexes, a steered molecular dynamics (SMD) simulation to determine a binding free energy of each protein-ligand complex; and determining, based at least on the binding free energy of each protein-ligand complex, a binding affinity between the ligand and the protein molecule.

In another aspect, there is provided a computer program product including a non-transitory computer readable medium storing instructions. The instructions may cause operations may executed by at least one data processor. The operations may include: identifying at least a portion of a binding site in a ligand forming an interface between the ligand and a protein molecule; generating one or more protein-ligand complexes by at least sampling a plurality of different poses of the ligand in a binding site in the protein molecule, the plurality of different poses being constrained by the binding site of the ligand such that each pose of the plurality of poses orients the binding site of the ligand to bind to the binding site of the protein molecule; performing, for each protein-ligand complex of the one or more protein-ligand complexes, a steered molecular dynamics (SMD) simulation to determine a binding free energy of each protein-ligand complex; and determining, based at least on the binding free energy of each protein-ligand complex, a binding affinity between the ligand and the protein molecule.

In some variations, one or more features disclosed herein including the following features can optionally be included in any feasible combination.

In some variations, the ligand may be another protein molecule having a sequence of amino acid residues and the binding site of the ligand may include a set of interfacial residues comprising one or more amino acid residues forming at least a portion of the interface between the ligand and the protein molecule.

In some variations, one or more amino acid residues for inclusion in the set of interfacial residues may be selected from a second plurality of amino acid residues forming the interface between the ligand and the protein molecule.

In some variations, a threshold quantity of amino acid residues from the second plurality of amino acid residues may be selected for inclusion in the set of interfacial residues

In some variations, the one or more amino acid residues may be selected randomly from the second plurality of amino acid residues.

In some variations, the one or more amino acid residues may be selected based on at least one of (i) a location of the one or more amino acid residues in the binding site of the ligand (ii) a location of the one or more amino acid residues in the ligand as a whole, (iii) a likelihood of the one or more amino acid residue participating in a binding interaction with the protein molecule, and (iv) a type of the one or more amino acid residues.

In some variations, the set of interfacial residues may be identified based at least on experimental data identifying one or more amino acid residues participating in a binding interaction between the ligand and the protein molecule.

In some variations, the ligand may be an antigen and the protein molecule may be an antibody or a T-cell receptor (TCR). The binding site of the ligand may include an epitope in the antigen recognized by the antibody or the T-cell receptor (TCR).

In some variations, the ligand may be an enzyme inhibitor and the protein molecule may be an enzyme.

In some variations, the ligand may be a small molecule having a plurality of atoms. The binding site of the ligand may include one or more atoms in the small molecule participating in a binding interaction with the protein molecule.

In some variations, the ligand may be a nucleic acid molecule and the binding site of the ligand may include one or more nucleotides in the nucleic acid molecule participating in a binding interaction with the protein molecule.

In some variations, an individual round of steered molecular dynamics (SMD) simulation may include dissociating the protein-ligand complex by at least pulling the ligand away from the protein molecule with a constant force over a period of time.

In some variations, an individual round of steered molecular dynamics (SMD) simulation may include dissociating the protein-ligand complex by at least pulling the ligand away from the protein molecule with a constant velocity over a period of time.

In some variations, a plurality of rounds of the steered molecular dynamics (SMD) simulation may be performed for each protein-ligand complex. The A quantity of work performed to dissociate the protein-ligand complex may be determined for each round of steered molecular dynamics (SMD) simulation. The binding free energy of each protein-ligand complex of the one or more protein-ligand complexes may be determined based at least on the quantity of work performed for each round of steered molecular dynamics (SMD) simulation.

In some variations, the binding free energy of each protein-ligand complex may correspond to an average of the quantity of work performed across the plurality of rounds of steered molecular dynamics (SMD) simulation.

In some variations, the quantity of work performed to dissociate the protein-ligand complex in each round of steered molecular dynamics (SMD) simulation may correspond a quantity of work performed to transition the protein-ligand complex from a bound state to an unbound state.

In some variations, the binding free energy of each protein-ligand complex may correspond to a difference between a first energy of the protein-ligand complex in a bound state and a second energy of the protein-ligand complex in an unbound state.

In some variations, one or more hyperparameters of the steered molecular dynamics (SMD) simulation may be adjusted.

In some variations, the one or more hyperparameters may be adjusted to reduce a difference between the binding free energy of a same protein-ligand complex across different rounds of steered molecular dynamics (SMD) simulation.

In some variations, the one or more hyperparameters may include a presence or absence of solvent molecules surrounding each protein-ligand complex, a rate at which each protein-ligand complex is pulled to dissociate the protein-ligand complex, a duration that each protein-ligand complex is pulled to dissociate each protein-ligand complex, and a quantity of rounds of steered molecular dynamics (SMD) simulation performed for each protein-ligand complex.

In some variations, a plurality of protein-ligand complexes may be determined by at least sampling the plurality of different poses of the ligand in the binding site of the protein molecule. The one or more protein-ligand complexes for performing the steered molecular dynamics (SMD) simulation may be selected from the plurality of protein-ligand complexes.

In some variations, the one or more protein-ligand complexes may be selected for performing the steered molecular dynamics (SMD) simulation based on at least one of (i) an energy of each protein-ligand complex of the plurality of protein-ligand complexes in a bound state; (ii) a shape complementarity between the ligand and the protein molecule in each protein-ligand complex, and (iii) a quantity of polar interactions across an interface between the ligand and the protein molecule in each protein-ligand complex.

In some variations, the generating the one or more protein-ligand complexes may include docking, to the protein molecule, the ligand in a first pose in which the ligand is oriented to bind to the protein molecule at least a threshold portion of the binding site of the ligand.

In some variations, the generating the one or more protein-ligand complexes may further include excluding, from being docked to the protein molecule, the ligand in a second pose in which the ligand is oriented to bind to the protein molecule at less than the threshold portion of the binding site in the ligand.

In some variations, the generating the one or more protein-ligand complexes may further include excluding, from being docked to the protein molecule, the ligand in a second pose in which the ligand is oriented to bind to the protein molecule at greater than a threshold portion of a non-binding site in the ligand

In some variations, each protein-ligand complex of the one or more protein-ligand complexes may include the ligand being bound to the protein molecule at greater than the threshold portion of the binding site in the ligand and/or less than the threshold portion of the non-binding site in the ligand.

In some variations, the binding site of the protein molecule may include at least a portion of (i) one or more antigen binding fragments (Fab) of the protein molecule, (ii) a variable domain (Fv) of the protein molecule, or (iii) one or more complementarity-determining regions (CDRs) of the protein molecule.

In some variations, the binding site of the protein molecule may be an active site where a substrate binds to the protein molecule or an allosteric site.

Systems, methods, and articles of manufacture, including computer program products, are also provided for identifying a protein-ligand complex that corresponds to the native pose, or the ground-truth docking pose, between the constituent ligand and protein molecule by at least performing one or more rounds of steered molecular dynamics (SMD) simulation to dissociate protein-ligand complexes formed through constrained docking. In one aspect, there is provided a system including at least one data processor and at least one memory. The at least one memory may store instructions, which when executed by the at least one data processor, result in operations comprising: identifying at least a portion of a binding site in a ligand forming an interface between the ligand and a protein molecule; generating a plurality of protein-ligand complexes by at least sampling a plurality of different poses of the ligand in a binding site of the protein molecule, the plurality of different poses being constrained by the binding site of the ligand such that each pose of the plurality of poses includes the ligand being oriented to bind to the protein molecule at the binding site; performing, for each protein-ligand complex of the plurality of protein-ligand complexes, a steered molecular dynamics (SMD) simulation to determine a binding free energy each protein-ligand complex; and identifying, based at least on the binding free energy of each protein-ligand complex, a protein-ligand complex of the plurality of protein-ligand complexes as exhibiting a ground-truth pose of the ligand docked in the binding site of the protein molecule.

In another aspect, there is provided a method for identifying a protein-ligand complex exhibiting the native pose, or the ground-truth docking pose, between a ligand and a protein molecule. The method may include: identifying at least a portion of a binding site in a ligand forming an interface between the ligand and a protein molecule; generating a plurality of protein-ligand complexes by at least sampling a plurality of different poses of the ligand in a binding site of the protein molecule, the plurality of different poses being constrained by the binding site of the ligand such that each pose of the plurality of poses includes the ligand being oriented to bind to the protein molecule at the binding site; performing, for each protein-ligand complex of the plurality of protein-ligand complexes, a steered molecular dynamics (SMD) simulation to determine a binding free energy each protein-ligand complex; and identifying, based at least on the binding free energy of each protein-ligand complex, a protein-ligand complex of the plurality of protein-ligand complexes as exhibiting a ground-truth pose of the ligand docked in the binding site of the protein molecule.

In another aspect, there is provided a computer program product including a non-transitory computer readable medium storing instructions. The instructions may cause operations may executed by at least one data processor. The operations may include: identifying at least a portion of a binding site in a ligand forming an interface between the ligand and a protein molecule; generating a plurality of protein-ligand complexes by at least sampling a plurality of different poses of the ligand in a binding site of the protein molecule, the plurality of different poses being constrained by the binding site of the ligand such that each pose of the plurality of poses includes the ligand being oriented to bind to the protein molecule at the binding site; performing, for each protein-ligand complex of the plurality of protein-ligand complexes, a steered molecular dynamics (SMD) simulation to determine a binding free energy each protein-ligand complex; and identifying, based at least on the binding free energy of each protein-ligand complex, a protein-ligand complex of the plurality of protein-ligand complexes as exhibiting a ground-truth pose of the ligand docked in the binding site of the protein molecule.

In some variations, one or more features disclosed herein including the following features can optionally be included in any feasible combination.

In some variations, the protein-ligand complex may be identified as corresponding to the ground-truth pose of the ligand docked in the binding site of the protein molecule based at least on the binding free energy of the protein-ligand complex satisfying one or more criteria.

In some variations, the one or more criteria may include the binding free energy of the protein-ligand complex satisfying one or more thresholds.

In some variations, the one or more criteria may include the protein-ligand complex having a lowest binding free energy within the plurality of protein-ligand complexes.

In some variations, the protein-ligand complex may be identified as exhibiting a greater correspondence to the ground-truth pose than the one or more other protein-ligand complexes based at least on the protein-ligand complex having a lower binding free energy than one or more other protein-ligand complexes in the plurality of protein-ligand complexes.

In some variations, a plurality of rounds of the steered molecular dynamics (SMD) simulation may be performed to determine the binding free energy of each protein-ligand complex. The binding free energy of each protein-ligand complex may correspond to an average of the quantity of work performed to dissociate each protein-ligand complex across the plurality of rounds of steered molecular dynamics (SMD) simulation for each protein-ligand complex.

In some variations, a subset of protein-ligand complexes including the protein-ligand complex may be selected from the plurality of protein-ligand complexes for performing the steered molecular dynamics (SMD) simulation.

In some variations, the subset of protein-ligand complexes may be selected based on at least one of (i) an energy of each protein-ligand complex of the plurality of protein-ligand complexes in a bound state; (ii) a shape complementarity between the ligand and the protein molecule in each protein-ligand complex, and (iii) a quantity of polar interactions across an interface between the ligand and the protein molecule in each protein-ligand complex.

In some variations, the ligand may be another protein molecule having a sequence of amino acid residues. The binding site of the ligand may include a set of interfacial residues comprising one or more amino acid residues forming at least a portion of the interface between the ligand and the protein molecule.

In some variations, the ligand may be a small molecule having a plurality of atoms. The binding site of the ligand may include one or more atoms in the small molecule participating in a binding interaction with the protein molecule.

In some variations, the binding site of the protein molecule may include at least a portion of (i) one or more antigen binding fragments (Fab) of the protein molecule, (ii) a variable domain (Fv) of the protein molecule, or (iii) one or more complementarity-determining regions (CDRs) of the protein molecule.

In some variations, the binding site of the protein molecule may be an active site where a substrate binds to the protein molecule or an allosteric site.

In some variations, the generating of the plurality of protein-ligand complexes may include docking, to the protein molecule, the ligand in a first pose in which the ligand is oriented to bind to the protein molecule at a threshold portion of the binding site in the ligand while excluding, from being docked to the protein molecule, at least one of (i) the ligand in a second pose in which the ligand is oriented to bind to the protein molecule at less than the threshold portion of the binding site in the ligand and (ii) the ligand in a third pose in which the ligand is oriented to bind to the protein molecule at greater than a threshold portion of one or more non-binding sites in the ligand.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g., the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to the binding interactions between two protein molecules, a protein molecule and a small molecule, and a protein molecule and a nucleic acid molecule, it should be readily understood that such features are not intended to be limiting. The claims that follow this disclosure are intended to define the scope of the protected subject matter.

### DESCRIPTION OF THE DRAWINGS

The accompanying drawings, which are incorporated in and constitute a part of this specification, show certain aspects of the subject matter disclosed herein and, together with the description, help explain some of the principles associated with the subject matter disclosed herein. The patent or application file contains at least one drawing executed in color. Copies of this patent or patent application publication with color drawings will be provided by the Office upon request and payment of the necessary fee. In the drawings,

FIG. 1 depicts a system diagram illustrating an example of a molecular analysis system 100, in accordance with some example embodiments;

FIG. 2A depicts a flowchart illustrating an example of a process for determining the binding affinity between a protein molecule and a ligand, in accordance with some example embodiments;

FIG. 2B depicts a flowchart illustrating an example of a process for determining the native pose of ligand docked in a binding site of a protein molecule, in accordance with some example embodiments;

FIG. 3 depicts a schematic diagram illustrating an example of two protein molecules undergoing constrained protein-ligand docking, in accordance with some example embodiments;

FIG. 4 depicts a schematic diagram illustrating a difference between conventional protein-ligand docking and constrained protein-ligand docking between two protein molecules, in accordance with some example embodiments;

FIG. 5 depicts a graph illustrating the work versus separation distance curves of multiple rounds of steered molecular dynamics simulation to dissociate a protein-ligand complex, in accordance with some example embodiments;

FIG. 6A depicts graphs illustrating steered molecular dynamics (SMD) simulation based identification of the native pose of a protein molecule bound to a ligand, in accordance with some example embodiments;

FIG. 6B depicts a graph comparing the binding free energy determined through steered molecular dynamics (SMD) simulations performed on protein-ligand complexes generated through binding-site constrained protein-ligand docking and the corresponding experimentally determined binding free energies, in accordance with some example embodiments;

FIG. 7 depicts graphs illustrating the effects of constraining protein-ligand docking to different quantities of interfacial amino acid residues, in accordance with some example embodiments;

FIG. 8 depicts graphs illustrating the work versus time curves for dissociating protein-ligand complexes containing binding protein molecules, weakly binding protein molecules, and non-binding protein molecules, in accordance with some example embodiments;

FIG. 9 depicts a graph illustrating a comparison between experimentally derived binding free energies and computationally derived binding free energies, in accordance with some example embodiments; and

FIG. 10 depicts a block diagram illustrating an example of a computing system, in accordance with some example embodiments.

When practical, similar reference numbers are used to refer to same or similar items in the drawings.

### DETAILED DESCRIPTION

The interaction between two molecules, such as the binding interaction between a protein molecule and a ligand (e.g., another protein molecule, a small molecule, a nucleic acid molecule, and/or the like), plays a significant role in a multitude of biological processes. Accordingly, understanding the dynamics of protein-ligand interactions is a salient part of drug discovery. For example, one aspect of analyzing the interaction between a protein molecule and a ligand includes identifying the native pose of the ligand bound to the protein molecule. In this context, the term “docked pose” refers to the pose assumed by the protein molecule and the ligand when the two molecules are in a bound state. While the protein molecule and the ligand may assume numerous different docked poses, the term “native pose” may refer to the docked pose that is most likely to be consistent with experimental observations. Another aspect of analyzing the interaction between a protein molecule and a ligand includes determining the binding affinity, or the strength of the binding interaction, between the two molecules. As described in more details below, the binding affinity between the protein molecule and the ligand may be quantified by the binding free energy of the two molecules, which is the change in free energy between the protein molecule and the ligand in a bound state and a second energy of the two molecules in an unbound state.

However, wet lab assessment of the binding interaction between a protein molecule and a ligand, such as surface plasmon resonance (SPR), can be prohibitively expensive for screening large numbers of protein molecules and ligands. Other wet lab techniques may yield a rudimentary binding determination between a ligand and a protein molecule (e.g., binder versus non-binder) but not a measure of the strength of the binding therebetween. For example, the output of X-ray crystallography is merely the shape complementarity between a protein molecule and a ligand, or the compatibility between the respective three-dimensional structures (or conformations). Thus, despite the high experimental cost of X-ray crystallography, further analysis is still required in order to determine the binding strength between the protein molecule and the ligand.

Meanwhile, existing computational techniques for determining binding affinity are also deficient for a number of reasons. For example, the binding affinity between the protein molecule and the ligand may vary depending on the pose (or orientation) of the ligand relative to the protein molecule. Furthermore, the binding affinity between the protein molecule and the ligand may also depend on shape complementarity, or the compatibility between the respective three-dimensional structure (or conformation) of the protein molecule and the ligand. Nevertheless, due to their flexible nature, the three-dimensional structure of each molecule participating in the binding interaction may evolve over time. Furthermore, the ligand may assume a near infinite number of possible poses (or orientations) relative to the protein molecule.

Conventional computational approaches to binding affinity analysis, which include a computationally intensive examination of every possible protein-ligand complex in silico to achieve an acceptable level of accuracy, is still too resource intensive to be a feasible alternative to wet lab screening. Given the foregoing inefficiencies, conventional wet lab and computational techniques are also ill suited for native pose analysis in which the binding affinity between a protein molecule and a ligand in a protein-ligand complex serves as a metric for assessing the correspondence between the protein-ligand complex and the native pose (or ground-truth pose) of the protein molecule bound to the ligand.

Various embodiments of the present disclosure overcome the limitations of conventional binding affinity and native pose analysis through a strategic reduction of the computational burden. For example, in some example embodiments, the computational burden associated with binding affinity and/or native pose analysis may be reduced by at least imposing, during the docking of a protein molecule and a ligand, one or more constraints such that a strategic subset of the possible protein-ligand complexes formed therefrom are generated for downstream binding affinity and/or native pose analysis. For example, in some cases, the native pose (or the ground-truth pose) of a ligand bound to a protein molecule and/or the binding affinity therebetween may be determined by evaluating the protein-ligand complexes formed by docking the protein molecule and the ligand in some but not all of the possible poses that can be assumed by the ligand. As noted, the ligand may assume a near infinite number of possible poses (or orientations) relative to the protein molecule. However, a stable protein-ligand complex may be formed when the protein molecule and the ligand both assume poses in which the protein molecule is able to interact with specific portions of the ligand identified as participating in the binding interaction between the ligand and the protein molecule. For instance, in some cases, one or more binding sites in the ligand at which binding interaction with the protein molecule is more likely to occur may be identified based on experimental data from, for example, mass spectrometry cross-linking, alanine scanning, epitope mapping, and/or the like. Accordingly, in some cases, downstream binding affinity and native pose analysis may be performed by evaluating those protein-ligand complexes in which the ligand is bound to at least a threshold portion of a binding site in the ligand and/or no greater than a threshold portion of a non-binding site in the ligand. Meanwhile, unstable protein-ligand complexes in which the ligand fails to bind to at least the threshold portion of the binding site in the ligand and/or is bound to more than the threshold portion of the non-binding site of the ligand may be excluded from downstream binding affinity and native pose analysis. Doing so may result in a strategic reduction of computational resources while maintaining the accuracy and precision of the downstream binding affinity and native pose analysis.

As used herein, the term “binding site” may refer to one or more portions of a molecule that participate in a binding interaction with another molecule. Meanwhile, those portions of the molecule that do not participate in the binding interaction with the other molecule may be referred to as the “non-binding site” of the molecule. For example, a protein-ligand complex may be formed as a result of a binding interaction between a binding site in the ligand and a binding site in the protein molecule. That binding interaction may exclude the non-binding sites in the ligand as well as the non-binding sites the protein molecule. In some cases, the ligand may be a small molecule, in which case the binding site may include one or more of the atoms forming the small molecule. Alternatively, in instances where the ligand is a nucleic acid molecule, the binding site may include one or more of the nucleotides forming the nucleic acid molecule. In instances where the ligand is another protein molecule having a sequence of amino acid residues, the binding site in the ligand may be a set of interfacial residues participating in the binding interaction with the protein molecule. In this context, the term “interfacial residue” may refer to an amino acid residue in the ligand that forms at least a portion of the interface between the ligand and the protein molecule. That is, an interfacial residue is an amino acid residue in the ligand that participate in the binding interaction with the protein molecule. Contrastingly, the term “non-interfacial residue” may refer to an amino acid residue in the ligand that does not participate in the binding with the protein molecule and is therefore not a part of the interface between the ligand and the protein molecule. In instances where the ligand is an antigen, for example, the protein molecule (e.g., an antibody, a T-cell receptor (TCR), and/or the like) may recognize and bind to a specific portion of the antigen called an epitope. Where the protein molecule is an antibody, the corresponding binding site (or paratope) in the protein molecule may include at least a portion of the antigen binding fragment (Fab), the variable domain (Fv), or the complementarity determining region (CDR) of the antibody.

Although the ligand may assume a near infinite number of poses relative to the protein molecule, not every possible pose may be relevant during protein-ligand docking because the protein molecule and the ligand cannot form a stable protein-ligand complex when the ligand is in certain poses. In particular, a protein-ligand complex is unlikely to be stable if the orientation of the ligand prevents a sufficient quantity of residues in the ligand identified as interfacial residues from participating in the binding interaction with the protein molecule. Alternatively and/or additionally, a protein-ligand complex is unlikely to be stable if the orientation of the ligand requires a greater than a threshold quantity of residues in ligand identified as non-interfacial residues to participate in the binding interaction with the protein molecule. Accordingly, those poses in which the ligand is oriented to bind to the protein molecule at less than a threshold portion of the binding site in the ligand and/or greater than a threshold portion of the non-binding site in the ligand may be immaterial at least because the protein molecule cannot interact with the non-binding site in the ligand is unlikely to form a stable protein-ligand complex. For example, in instances where the ligand is another protein molecule, a stable protein-ligand complex is unlikely to result when the ligand is oriented to bind to the protein molecule at fewer than a threshold quantity of interfacial residues and/or greater than a threshold quantity of non-interfacial residues. Avoiding poses where the corresponding protein-ligand complexes fail to exhibit a threshold likelihood of being stable during protein-ligand docking and excluding these protein-ligand complexes from further evaluation may significantly reduce the computational burden associated with determining the native pose and/or the binding affinity between the protein molecule and the ligand while still maintaining the accuracy of the results.

In some example embodiments, the binding affinity or the strength of the binding interaction between the protein molecule and the ligand may be determined based on the binding free energy of the individual protein-ligand complexes in a subset of the possible protein-ligand complexes formed by the protein molecule and the ligand. As noted, the binding free energy (ΔG) of a protein-ligand complex may correspond to a change in free energy between a first energy of the constituent protein molecule and ligand in a bound state (e.g., the first energy of the corresponding protein-ligand complex) and a second energy of the protein molecule and the ligand in an unbound state. Accordingly, in some cases, the binding free energy of the protein-ligand complex may be determined based at least on a quantity of work performed to dissociate the protein-ligand complex, for example, by transitioning the protein molecule and the ligand from a bound state in which the two molecules are separated by a first distance to an unbound state in which the two molecules are separated by a second distance that is greater than the first distance. Although there is some correlation between the energy of the protein-ligand complex in just the bound state and the binding affinity between the constituent protein molecule and ligand, the binding free energy of the protein-ligand complex may be a more accurate indicator of the binding affinity between the two molecules. Nevertheless, significant computational resources may be required to determine the binding free energy between a protein molecule and a ligand. For example, in some cases, the binding free energy of a protein-ligand complex may be determined based on the quantity of work performed to dissociate the protein-ligand complex during one or more rounds of steered molecular dynamics (SMD) simulation. As described in more detail below, the computational burden associated with determining the binding free energy between the protein molecule and the ligand may be reduced by performing steered molecular dynamics (SMD) simulation on a strategic subset of the possible protein-ligand complexes formed by the protein molecule and the ligand.

Alternatively and/or additionally, the binding free energy of a protein-ligand complex may be indicative of the degree of correspondence between the protein-ligand complex and the native pose (or ground-truth pose) of the constituent protein molecule bound to the ligand. As noted, the binding free energy of the protein-ligand complex may correspond to the quantity of work performed to dissociate the protein-ligand complex, for example, during one or more rounds of steered molecular dynamics (SMD) simulation. Thus, in some cases, the native pose (or ground-truth pose) of the protein molecule bound to the ligand, or the degree of correspondence between a corresponding protein-ligand complex and the native pose (or ground-truth pose), may be determined based on the quantity of work performed to dissociate individual protein-ligand complexes. For example, in some cases, a first protein-ligand complex may be identified, based at least on a first quantity of work performed to dissociate the first protein-ligand complex over one or more rounds of steered molecular dynamics (SMD) simulation, as corresponding to the native pose (or ground-truth pose) of the protein molecule bound to the ligand. Alternatively and/or additionally, the first protein-ligand complex may be identified, further based at least on a second quantity of work performed to dissociate a second protein-ligand complex (e.g., over one or more rounds of steered molecular dynamics (SMD) simulation), as exhibiting a greater correspondence to the native pose (or ground-truth pose) than the second protein-ligand complex if a greater quantity of work is performed to dissociate the first protein-ligand complex than the second protein-ligand complex. In this context, the terms “native pose” and “ground truth pose” may be used interchangeably to refer to a docked pose that is most likely to be consistent with experimental observations (e.g., X-ray crystallography) of the pose assumed by the protein molecule and the ligand when the protein molecule is bound to the ligand. As described in more detail below, the computational burden associated with native pose analysis may be reduced by at least by performing steered molecular dynamics (SMD) simulation to determine the binding free energies of a strategic subset of the possible protein-ligand complexes formed by constrained docking between the protein molecule and the ligand.

In some example embodiments, the computational burden associated with binding affinity and native analysis may be reduced by generating the subset of possible protein-ligand complexes to exclude those in which the ligand is oriented to bind to the protein molecule at fewer than a threshold quantity of interfacial residues and/or greater than a threshold quantity of non-interfacial residues. For example, in some cases, the subset of possible protein-ligand complexes may be generated by sampling, during protein-ligand docking, some but not all of the possible poses of the ligand. In particular, the poses sampled during protein-ligand docking may be constrained based on the binding site and/or the non-binding site in the ligand. For instance, depending on whether the ligand is another protein molecule, a small molecule, or a nucleic acid molecule, the poses sampled during protein-ligand docking may be constrained based on one or more of the amino acid residues (or interfacial residues), atoms, or nucleotides in the ligand that participate in the binding between the ligand and the protein molecule. In some cases, the binding site in the ligand (e.g., the interfacial residues, atoms, nucleotides, and/or the like) may be identified based on experimental data (e.g., mass spectrometry cross-linking, alanine scanning, epitope mapping, and/or the like.). Imposing this constraint may limit the poses that are sampled during protein-ligand docking to those in which the ligand is oriented to bind to the protein molecule at greater than a threshold portion of the binding site and/or less than a threshold portion of the non-binding site in the ligand. Otherwise, as noted, the ligand may assume a near infinite number of different poses (or orientations) relative to the protein molecule. Since at least some of these possible poses are immaterial due to the instability of the protein-ligand complexes formed therefrom, including these protein-ligand complexes in subsequent binding affinity and/or native pose evaluation may impose a significant but superfluous computational burden. This excessive computational burden may be eliminated by avoiding, during protein-ligand docking, poses in which the orientation of the ligand prevent a threshold portion of a binding site in the ligand from participating in a binding interaction with a binding site in the protein molecule.

In some example embodiments, in addition to imposing the aforementioned constraints to exclude certain poses of the ligand from being sampled during protein-ligand docking, the computational burden associated with binding affinity and/or native pose evaluation may be further reduced by excluding one or more of the protein-ligand complexes generated by the constrained protein-ligand docking. As noted, in some cases, protein-ligand docking may be constrained based on at least the binding site and/or the non-binding site in the ligand in order to generate a subset of possible protein-ligand complexes, which excludes those protein-ligand complexes in which the ligand is oriented to bind to the protein molecule at less than a threshold portion of the binding site and/or greater than a threshold portion of the non-binding site in the ligand. In some cases, some but not all of the protein-ligand complexes generated by the constrained protein-ligand docking may be selected to undergo further binding affinity and/or native pose evaluation based at least on the respective energies of each protein-ligand complex generated by the constrained protein-ligand docking. For example, in some cases, the one or more protein-ligand complexes may be selected based at least on an energy of each protein-ligand complex in a bound state. In some cases, the energy of a protein-ligand complex in a bound state may be estimated based on the energy of atomic interactions (e.g., electrostatic interactions, van der Waals interactions, and/or the like) present in the protein-ligand complex. Alternatively and/or additionally, the one or more protein-ligand complexes may be selected based on a shape complementarity between the ligand and the protein molecule in each protein-ligand complex and/or a quantity of polar interactions across an interface between the ligand and the protein molecule in each protein-ligand complex. As described in more details below, each of the protein-ligand complexes selected for further binding affinity and/or native pose evaluation may undergo multiple rounds of steered molecular dynamics (SMD) simulation. Accordingly, excluding at least some protein-ligand complexes that are energetically and/or structurally unfavorable from undergoing multiple rounds of steered molecular dynamics (SMD) simulation may further reduce the computational burden associated with the downstream binding affinity and/or native pose evaluation.

In some example embodiments, the binding free energy of each protein-ligand complex may be determined by performing round(s) of steered molecular dynamics (SMD) simulation. For example, in some cases, each round of steered molecular dynamics (SMD) simulation performed for an individual protein-ligand complex may include dissociating the protein-ligand complex by at least pulling the ligand away from the protein molecule while the protein-ligand complex is exposed to identical external conditions such as a constant pulling force, a constant pulling velocity, and/or the like. In this context, the act of “pulling” may refer to applying a force (e.g., a simulated force) to act on either or both of the protein molecule and the ligand. This force may displace one or both of the protein molecule and the ligand such that the protein-ligand complex is transitioned from a bound state (e.g., an initial state in which the protein molecule and the ligand are separated by a first distance) to an unbound state (e.g., a final state in which the protein molecule and the ligand are separated by a second distance). Moreover, the application of this force may result in a transfer of energy known as work. The amount of work that is performed may correspond to the amount of energy that is transferred in order to separate the protein molecule from the ligand. Accordingly, in some cases, the amount of work required to separate the protein molecule from the ligand may correspond to the binding free energy of the protein-ligand complex. That is, in some cases, the binding free energy for the protein-ligand complex may be determined based on the quantity of work performed to dissociate the protein-ligand complex in each round of steered molecular dynamics (SMD) simulation. It should be appreciated that during each round of steered molecular dynamics (SMD) simulation, the dissociation of the protein-ligand complex may be tantamount to transitioning the protein-ligand complex from a bound state (e.g., an initial state in which the protein molecule and the ligand are separated by a first distance) to an unbound state (e.g., a final state in which the protein molecule and the ligand are separated by a second distance) over a trajectory of intermediate states. The quantity of work performed to dissociate the protein-ligand complex may vary across multiple rounds of steered molecular dynamics (SMD) simulation even when the protein-ligand complex is subject to identical external conditions (e.g., pulling force, pulling velocity, and/or the like) at least because the aforementioned transition cannot be made sufficiently slow to ensure that the protein-ligand complex reaches thermodynamic equilibrium at every intermediate state. Accordingly, in some cases, the binding free energy of the protein-ligand complex may correspond to an average of the quantity of work performed across a threshold quantity of steered molecular dynamics (SMD) simulation rounds in which the protein-ligand complex is subject to identical external conditions including, for example, a constant pulling force, a constant pulling velocity, and/or the like.

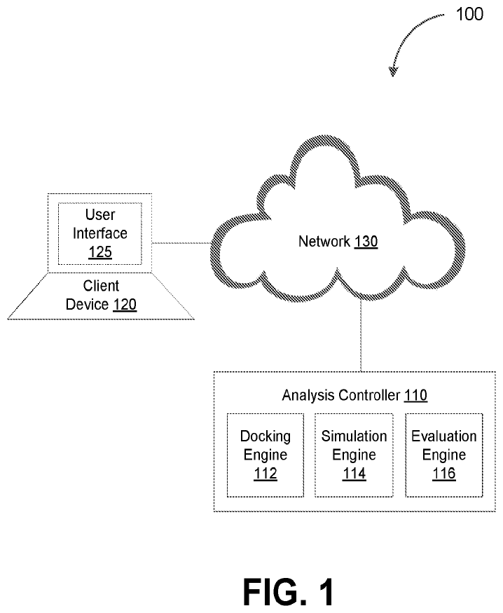

FIG. 1 depicts a system diagram illustrating an example of a molecular analysis system 100, in accordance with some example embodiments. Referring to FIG. 1, the molecular analysis system 100 may include an analysis controller 110 that is communicatively coupled with a client device 120 via a network 130. The client device 120 may be a processor-based device including, for example, a workstation, a desktop computer, a laptop computer, a high-performance computer, a smartphone, a tablet computer, a wearable apparatus, and/or the like. The network 130 may be a wired network and/or a wireless network including, for example, a local area network (LAN), a virtual local area network (VLAN), a wide area network (WAN), a public land mobile network (PLMN), the Internet, and/or the like.

Referring again to FIG. 1, the analysis controller 110 may determine the binding affinity between a protein molecule (e.g., an antibody, a T-cell receptor (TCR), an enzyme, and/or the like) and a ligand (e.g., a viral antigen, a tumor antigen, an enzyme-inhibitor, a small molecule, a nucleic acid molecule, and/or the like). For example, in some cases, the analysis controller 110 may determine the native pose (or ground-truth pose) of the protein molecule bound to the ligand and/or the binding affinity therebetween by at least performing an in silico evaluation of various protein-ligand complexes formed by the protein molecule and the ligand. However, because the ligand may assume a near infinite number of different poses in a binding site of the protein molecule, including many that results in unstable protein-ligand complexes because the ligand is oriented to bind to the protein molecule at a non-binding site in the ligand, in silico evaluation of every possible protein-ligand complex may impose an excessive computational burden. Accordingly, as described in more detail below, the computational burden associated with native pose and/or binding affinity evaluation may be reduced by at least limiting such downstream evaluation to a strategic subset of the possible protein-ligand complexes formed by the protein molecule and the ligand.

In some example embodiments, the computational burden associated with downstream evaluations to identify the native pose (or ground-truth pose) of the protein molecule bound to the ligand and/or the binding affinity therebetween may be reduced, by at least excluding those protein-ligand complexes in which the ligand is oriented to bind to the protein molecule at less than a threshold portion of the binding site and/or greater than a threshold portion of the non-binding site in the ligand. For example, in some cases, such protein-ligand complexes may be excluded by at least avoiding or deprioritizing, during protein-ligand docking, poses in which the ligand is oriented to bind to the protein molecule at less than the threshold portion of the binding site residues and/or greater than the threshold portion of the non-binding site in the ligand.

Referring again to FIG. 1, the analysis controller 110 may include a docking engine 112 that performs protein-ligand docking by at least sampling various different poses of the ligand bound to the protein molecule. In some cases, the docking engine 112 may constrain, based on at least the binding site and/or the non-binding site in the ligand, the poses that are sampled during protein-ligand docking. Accordingly, in some cases, the docking engine 112 may perform protein-ligand docking to generate a subset of possible protein-ligand complexes that includes those protein-ligand complexes in which the ligand is oriented to bind to the protein molecule at greater than the threshold portion of the binding site and/or less than the threshold portion of the non-binding site in the ligand. This subset of possible protein-ligand complexes may further exclude protein-ligand complexes in which the ligand is oriented to bind to the protein molecule at less than the threshold portion of the binding site and/or greater than the threshold portion of the non-binding site in the ligand.

The computational burden associated with various downstream evaluations, such as steered molecular dynamics (SMD) simulation to identify the native pose (or ground-truth pose) of the protein molecule bound to the ligand and/or to determine the binding affinity therebetween, may be reduced by avoiding protein-ligand complexes in which the ligand is oriented to bind to the protein molecule at less than the threshold portion of the binding site and/or greater than the threshold portion of the non-binding site in the ligand. Furthermore, these ligand-protein complexes, which are unstable due to the misalignment between a binding site in the ligand and a binding site in the protein molecule, may introduce outlying or anomalous values in downstream evaluations. As such, in some cases, in addition to reducing computational burden, the exclusion of ligand-protein complexes in which the ligand is not oriented to bind to the protein molecule at greater than the threshold portion of the binding site and/or less than the threshold portion of the non-binding site in the ligand may also increase the precision and accuracy of downstream analysis.

In some example embodiments, the computational burden associated with downstream evaluations, such as steered molecular dynamics (SMD) simulation for binding affinity and/or native pose evaluation, may be further reduced by excluding one or more of the protein-ligand complexes generated by the constrained protein-ligand docking. For example, in some cases, a protein-ligand complex generated by the constrained protein-ligand docking may be excluded based at least on the energy of the protein-ligand complex in a bound state failing to satisfy one or more thresholds. It should be appreciated that even when the ligand is oriented to bind to the protein molecule at least a threshold portion of the binding site in the ligand and/or no greater than a threshold portion of the non-binding site in the ligand, the resulting protein-ligand complex may not be sufficiently stable to warrant further downstream analysis. The instability of a protein-ligand complex may be indicated by the protein-ligand complex exhibiting an excessively high energy in a bound state, which may be estimated based on the energy of various atomic interactions present in the protein-ligand complex including, for example, electrostatic interactions, van der Waals interactions, and/or the like. Alternatively and/or additionally, the protein-ligand complex may be identified as unstable based on a shape complementarity between the ligand and the protein molecule in the protein-ligand complex and/or the quantity of polar interactions across an interface between the ligand and the protein molecule in the protein-ligand complex. Accordingly, this particular exclusion may further reduce the computational burden of downstream evaluations by at least omitting, from the subset of possible protein-ligand complexes that undergo steered molecular dynamics (SMD) simulation, one or more protein-ligand complexes that are energetically and/or structurally unfavorable even when these protein-ligand complexes are formed by the ligand oriented to bind to the protein molecule at least a threshold portion of the binding site in the ligand and/or no greater than a threshold portion of the non-binding site in the ligand.

In some example embodiments, the native pose (or ground-truth pose) of the protein molecule bound to the ligand and/or the binding affinity therebetween may be determined based at least on the binding free energy of each protein-ligand complex in the subset of possible protein-ligand complexes generated by the docking engine 112. For example, the binding free energy of a protein-ligand complex may correspond to a difference between a first energy of the protein-ligand complex in a bound state in which the protein molecule and the ligand are separated by a first distance and a second energy of the protein-ligand complex in which the protein molecule and the ligand are separated by a second distance that is greater than the first distance. In some cases, the binding free energy of the protein-ligand complex may correspond to the quantity of work required to dissociate the protein-ligand complex, for example, by transitioning the protein-ligand complex from the bound state to the unbound state over a trajectory of intermediate states.

In some example embodiments, the quantity of work required to dissociate the protein-ligand complex may be determined by performing multiple rounds of steered molecular dynamics (SMD) simulation, each of which dissociating the protein-ligand complex by subjecting the protein-ligand complex to identical external conditions such as a constant pulling force, a constant pulling velocity, and/or the like. For example, as shown in FIG. 1, the analysis controller 110 may include a simulation engine 114 that performs, for each protein-ligand complex generated by the docking engine 112, multiple rounds of steered molecular dynamics (SMD) simulation. Moreover, the analysis controller 110 may include an evaluation engine 116 that performs, based at least on the quantity of work performed to dissociate the protein-ligand complex in each round of steered molecular dynamics (SMD) simulation, one or more downstream evaluations. For instance, in some cases, the evaluation engine 116 may determine, based at least on the quantity of work performed to dissociate the protein-ligand complex in each round of steered molecular dynamics (SMD) simulation, the binding free energy of the protein-ligand complex. Moreover, in some cases, the evaluation engine 116 may determine, based at least on the binding free energy of each protein-ligand complex in the subset of possible protein-ligand complexes generated by constrained protein-ligand docking, the binding affinity between the constituent protein molecule and the ligand. Alternatively and/or additionally, the evaluation engine 116 may identify, based at least on the binding free energy of each protein-ligand complex in the subset of protein-ligand complexes generated by constrained protein-ligand docking, one or more protein-ligand complexes as corresponding to the native pose (or ground-truth pose) of the protein molecule bound to the ligand.

As noted, in some cases, identical external conditions (e.g., a constant pulling force, a constant pulling velocity, and/or the like) may be imposed across multiple rounds of steered molecular dynamics (SMD) simulation performed for a protein-ligand complex. Nevertheless, the quantity of work performed to dissociate the protein-ligand complex may still vary across different rounds of steered molecular dynamics (SMD) simulation at least because the transition of the protein-ligand complex from the bound state to the unbound state cannot be made sufficiently slow during each round of steered molecular dynamics (SMD) simulation to ensure that the protein-ligand complex reaches thermodynamic equilibrium at every intermediate state. Accordingly, in some cases, the binding free energy of an individual protein-ligand complex may correspond to an average quantity of the work performed to dissociate the protein-ligand complex across multiple rounds of steered molecular dynamics (SMD) simulation. Moreover, in some cases, one or more hyperparameters of the steered molecular dynamics (SMD) simulation may be adjusted in order to reduce or minimize the variations in the quantity of work performed to dissociate the same protein-ligand complex across different rounds of steered molecular dynamics (SMD) simulation. For example, in some cases, the variation in the quantity of work performed across different rounds of steered molecular dynamics (SMD) simulation may be reduced by at least reducing the rate at which the protein-ligand complex is pulled to dissociate the protein-ligand complex, thus slowing the transition of the protein-ligand complex from the bound state to the unbound state. Other examples of hyperparameters that may be adjusted (or tuned) include the presence or absence of solvent molecules surrounding the protein-ligand complex, the duration that the protein-ligand complex is pulled to dissociate the protein-ligand complex, the quantity of rounds of steered molecular dynamics (SMD) simulation performed for the protein-ligand complex, and/or the like.

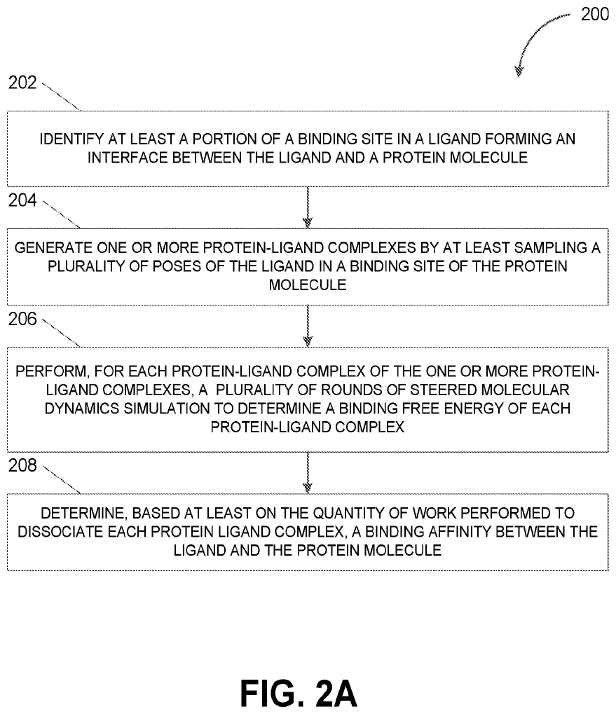

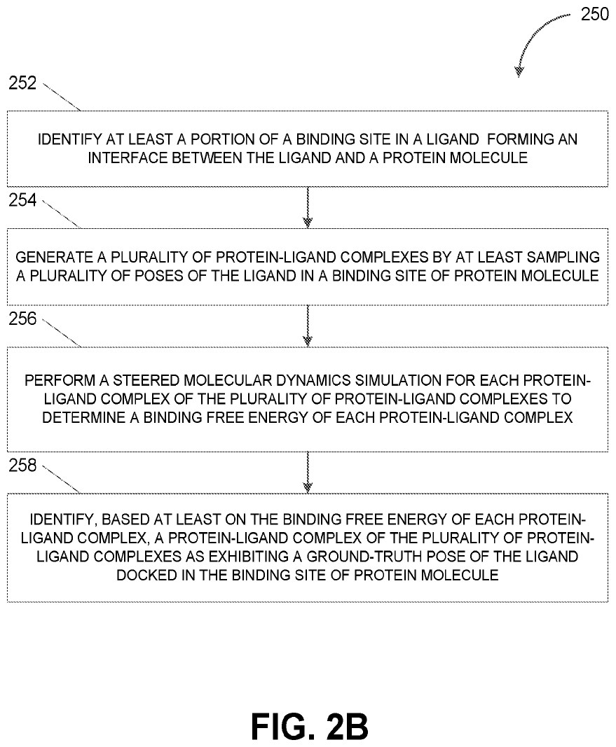

As noted, in some example embodiments, the analysis controller 110 may determine the binding affinity between a protein molecule and a ligand by at least performing one or more rounds of steered molecular dynamics (SMD) simulation on a subset of possible protein-ligand complexes generated by constrained protein-ligand docking. An example of the process for determining the binding affinity between a protein molecule and a ligand is shown in FIG. 2A. In some cases, the analysis controller 110 may also determine the native pose (or ground-truth pose) assumed by the protein molecule and the ligand in a bound state by at least performing one or more rounds of steered molecular dynamics (SMD) simulation on a subset of possible protein-ligand complexes generated by constrained protein-ligand docking. An example of the process for determining the native pose (or ground-truth pose) of a protein molecule bound to a ligand is shown in FIG. 2B.

FIG. 2A depicts a flowchart illustrating an example of a process 200 for determining the binding affinity between a protein molecule and a ligand, in accordance with some example embodiments. Referring to FIGS. 1 and 2A, the process 200 may be performed by the analysis controller 110, for example, by the docking engine 112, the simulation engine 114, and the evaluation engine 116. In some cases, the process 200 may be performed to determine the binding affinity between a protein molecule and a ligand. As described in more details below, one or more rounds of steered molecular dynamics (SMD) simulation may be performed to determine the binding affinity between the protein molecule and the ligand. However, instead of performing steered molecular dynamics (SMD) simulations on every possible protein-ligand complex, the process 200 may reduce the computational burden of the analysis by at least identifying a strategic subset of possible protein-ligand complexes, such as those protein-ligand complexes in which the ligand is oriented to bind to the protein molecule at more than a threshold portion of the binding site in the ligand and/or less than a threshold portion of the non-binding site in the ligand.

At 202, at least a portion of a binding site in a ligand forming an interface between the ligand and a protein molecule may be identified, e.g., by the analysis controller 110. In some example embodiments, the binding site in the ligand forming interface between the ligand and the protein molecule may be identified in order for the binding site in the ligand to serve as a constraint for constrained protein-ligand docking. For example, in some cases, a strategic subset of the possible protein-ligand complexes formed by the protein molecule bound to the ligand may be identified based on the binding site in the ligand for subsequent binding affinity evaluation. For example, in some cases, at least a portion of the binding site in the ligand may be identified based on experimental data, such as mass spectrometry cross-linking, alanine scanning, epitope mapping, and/or the like. Depending on the type of the ligand, the binding site in the ligand may include a set of interfacial residues, atoms, and/or nucleotides forming at least a portion of the interface between the ligand and the protein molecule.

In some cases, imposing the binding site constraint may reduce the computational burden of the downstream binding affinity analysis by at least excluding one or more unstable protein-ligand complexes from downstream binding affinity analysis. As noted, a protein-ligand complex in which the ligand is oriented to bind to the protein molecule at less than a threshold portion of the binding site in the ligand and/or greater than a threshold portion of the non-binding site in the ligand is an unstable structure. Including protein-ligand complexes that lack structural stability in binding affinity evaluation, which may include multiple rounds of computationally intense steered molecular dynamics (SMD) simulation, can impose unnecessary computational burden. Accordingly, in some cases, instead of sampling every possible pose of the ligand in a binding site of the protein molecule, poses in which the ligand is oriented to bind to the protein molecule at less than a threshold portion of the binding site and/or greater than a threshold portion of the non-binding site in the ligand may be avoided during protein-ligand docking. For example, in instances where the ligand is another protein molecule, protein-ligand docking may be constrained by the set of interfacial residues in the ligand, meaning that poses in which the ligand is oriented to bind to the protein molecule at fewer than a threshold quantity of interfacial residues and/or greater than a threshold quantity of non-interfacial residues in the ligand may be avoided during protein-ligand docking. As such, the resulting set of protein-ligand complexes exclude those in which the ligand is oriented to bind to the protein molecule at less than the threshold portion of the binding site (e.g., less than a threshold quantity of interfacial residues, atoms, nucleotides, and/or the like) and/or greater than the threshold portion of the non-binding site (e.g., greater than a threshold quantity of non-interfacial residues, atoms, nucleotides, and/or the like).

In some example embodiments, the binding site in the ligand may include a set of interfacial residues, atoms, or nucleotides forming at least a portion of the interface between the ligand and the protein molecule. In some cases, the binding site in the ligand may include a threshold quantity of interfacial residues, atoms, and/or nucleotides identified, for example, based on experimental data such as mass spectrometry cross-linking, alanine scanning, epitope mapping, and/or the like. For example, in instances where the ligand is another protein molecule, the set of interfacial residues forming the binding site may include a minimum quantity of amino acid residues (e.g., 2 amino acid residues and/or the like) forming the interface between the ligand and the protein molecule. Alternatively and/or additionally, the set of interfacial residues forming the binding site may include a maximum quantity of amino acid residues (e.g., 6 amino acid residues and/or the like). It should be appreciated that the quantity of interfacial residues, atoms, or nucleotides included as a part of the binding site in the ligand may be adjusted in order to balance computational complexity and accuracy. Moreover, in some cases, the binding site in the ligand may include some but not all of the interfacial residues, atoms, and/or nucleotides at the interface between the ligand and the protein molecule. Instead, in some cases, a threshold quantity of amino acid residues, atoms, or nucleotides at the interface between the ligand and the protein molecule may be selected for inclusion in the binding site. For instance, where the ligand is another protein molecule, the binding site in the ligand may include a random selection of the amino acid residues at the interface between the ligand and the protein molecule. Alternatively and/or additionally, one or more amino acid residues may be selected based on one or more (i) a location of the interfacial residues the binding site of the ligand (e.g., to favor amino acid residues located toward a center of the interface), (ii) a location of the one or more amino acid residues in the ligand as a whole (e.g., to favor amino acid residues located in a complementarity determining region (CDR)), (iii) a likelihood of the one or more amino acid residue participating in a binding interaction with the protein molecule, and (iv) a type of the one or more amino acid residues.

At 204, one or more protein-ligand complexes are generated by at least sampling a plurality of poses of the ligand in a binding site in the protein molecule. In some example embodiments, binding site constrained protein-ligand docking may be performed by at least sampling a variety of poses of the ligand in a binding site of the protein molecule, thus generating a subset of some but not all of the possible protein-ligand complexes formed by the ligand and the protein molecule for evaluating the binding affinity between the ligand and the protein molecule. As noted, in some cases, the binding site constraint may include the set of interfacial residues, atoms, or nucleotides in the ligand that forms at least a portion of the interface between the ligand and the protein molecule. Imposing the docking site constraint may exclude, during protein-ligand docking, the sampling of one or more poses of the ligand in which the ligand is oriented to bind to the protein molecule at less than a threshold portion of the binding site (e.g., fewer than a threshold quantity of interfacial residues, atoms, nucleotides, and/or the like) and/or greater than a threshold portion of the non-binding site in the ligand (e.g., greater a threshold quantity of non-interfacial residues, atoms, nucleotides, and/or the like). As such, the protein-ligand complexes that are generated by the binding-site constrained protein-ligand docking may exclude protein-ligand complexes in which the ligand is oriented to bind to the protein molecule at less than a threshold portion of the binding site (e.g., fewer than a threshold quantity of interfacial residues, atoms, nucleotides, and/or the like) and/or greater than a threshold portion of the non-binding site in the ligand (e.g., greater a threshold quantity of non-interfacial residues, atoms, nucleotides, and/or the like). The instability of such protein-ligand complexes means that excluding these protein-ligand complexes from binding affinity evaluation, which may include multiple rounds of computationally intense steered molecular dynamics (SMD) simulation to determine the work required to dissociate each protein-ligand complex, obviates unnecessary computational burden.

At 206, a steered molecular dynamics (SMD) simulation are performed for each protein-ligand complex of the one or more protein-ligand complexes to determine a binding free energy of each protein-ligand complex. In some example embodiments, multiple rounds of steered molecular dynamics (SMD) simulation may be performed for each protein-ligand complex generated by the binding site constrained protein-ligand docking performed at operation 204. Alternatively, in some cases, a further subset of the protein-ligand complexes generated at operation 204 may be selected, for example, based at least on the bound state energy, shape complementarity, and/or quantity of polar interactions of each protein-ligand complex, to undergo multiple rounds of steered molecular dynamics (SMD) simulation. Doing so may further reduce the computational burden of the binding affinity analysis.

In some cases, the steered molecular dynamics (SMD) simulation may be performed in accordance with one or more hyperparameters including, for example, a presence or absence of solvent molecules surrounding each protein-ligand complex, a rate at which each protein-ligand complex is pulled to dissociate the protein-ligand complex, a duration that each protein-ligand complex is pulled to dissociate the protein-ligand complex, a quantity of rounds of steered molecular dynamics (SMD) simulation performed for each protein-ligand complex, and/or the like. In some cases, the one or more hyperparameters of the steered molecular dynamics (SMD) simulation may be adjusted to reduce or minimize variations in results across different rounds of steered molecular dynamics (SMD) simulation performed for the same protein-ligand complex.

In some cases, a single round of steered molecular dynamics (SMD) simulation may include dissociating the protein-ligand complex by pulling the ligand away from the protein molecule. In some cases, the protein-ligand complex may be subjected identical external conditions during each round of steered molecular dynamics (SMD) simulation. For example, in some cases, each round of steered molecular dynamics (SMD) simulation may include the ligand being pulled from the protein molecule with a constant force or at a constant velocity. The binding free energy of the protein-ligand complex may be determined based at least on the quantity of work that is performed during each round of steered molecular dynamics (SMD) simulation to dissociate the ligand from the protein molecule. For instance, the binding free energy of the protein-ligand complex, which corresponds to a difference in a first energy of the protein-ligand complex in a bound state and a second energy of the protein-ligand complex in an unbound state, may be determined based on Equation (1) below.

$${e}^{- \frac{Δ G}{kT}} = \overline{{e}^{- \frac{W}{kT}}} \qquad ( 1 )$$

wherein ΔG denotes the free energy difference between the bound and unbound states of the protein-ligand complex, W denotes the work performed to dissociate the protein-ligand complex, k denotes the Boltzmann constant, and T denotes the temperature of the protein-ligand complex in its initial bound state.

According to Equation (1), the binding free energy of the protein-ligand complex may be determined based on the work that is performed to dissociate the protein-ligand complex across multiple rounds of steered molecular dynamics (SMD) simulations. That is, in some cases, a threshold quantity of rounds of steered molecular dynamics (SMD) simulations may be performed because the dissociation of the protein-ligand complex is a nonequilibrium process in which the protein-ligand complex transitions from the equilibrium bound state to a nonequilibrium unbound state over a succession of nonequilibrium intermediate states. Thus, even when the protein-ligand complex is subjected to identical external conditions during each round of steered molecular dynamics (SMD) simulation, the protein-ligand complex may nevertheless dissociate over a different trajectory such that a different quantity of work is performed during each round of steered molecular dynamics (SMD) simulation.

At 208, a binding affinity between the ligand and the protein molecule is determined based at least on the binding free energy of each protein-ligand complex. In some example embodiments, the binding affinity between the protein molecule and the ligand may be determined based at least on the binding free energy of stable protein-ligand complexes, which are those protein-ligand complexes in which the ligand is oriented to bind to the protein molecule at more than a threshold portion of the binding site in the ligand and/or less than a threshold portion of the non-binding site in the ligand. As noted, stable protein-ligand complexes may be identified through protein-ligand docking constrained based on at least a portion of the binding site in the ligand (e.g., one or more interfacial residues, atoms, or nucleotides in the ligand that participate in the binding between the ligand and the protein molecule). Binding site constrained protein-ligand docking avoids sampling poses in which the ligand is oriented to bind to the protein molecule at less than a threshold portion of the binding site and/or greater than a threshold portion of the non-binding site in the ligand, thus preventing the generation of unstable protein-ligand complexes for subsequent binding affinity analysis. In other words, the result of binding site constrained protein-ligand docking may be a subset of the possible protein-ligand complexes formed by the ligand bound to the protein molecule that excludes unstable protein-ligand complexes.

In some example embodiments, one or more rounds of steered molecular dynamics (SMD) simulations may be performed to determine the binding free energies of each protein-ligand complex by at least determining the quantity of work performed to dissociate each protein-ligand complex. Since the binding site constrained docking excludes unstable protein complexes, the binding affinity between the protein molecule and the ligand may be determined based on the binding free energies of protein-ligand complexes in which the ligand is oriented to bind to the protein molecule at more than a threshold portion of the binding site in the ligand and/or less than a threshold portion of the non-binding site in the ligand. In some cases, the binding affinity between the protein molecule and the ligand may be determined based on the binding free energies of multiple protein-ligand complexes (e.g., multiple stable protein-ligand complexes and not unstable protein-ligand complexes). For example, in some cases, the binding affinity between the protein molecule and the ligand may be determined based on a mean, a mode, a median, a maximum, a minimum, and/or a range of the binding free energy of the different protein-ligand complexes generated by binding site constrained protein-ligand docking. In some cases, at least some variability may be present in the binding free energies of the different protein-ligand complexes determined by the steered molecular dynamics (SMD) simulations. For example, this variability may be present across the binding free energies determined by different rounds of steered molecular dynamics (SMD) simulation performed on the same protein-ligand complex as well as across the binding free energies determined by the different rounds of steered molecular dynamics (SMD) simulation performed on different ligand protein-complexes of the same ligand and protein molecule. It should be appreciated that the variability (or the distribution) of the binding free energies may indicate a confidence in the binding affinity between the ligand and the protein molecule determined thereupon.

In some example embodiments, the binding free energy (ΔG) of a protein-ligand complex may serve as a binary classifier indicative of whether the ligand and the protein molecule exhibits sufficient binding affinity. For example, in some cases, where the binding free energy (ΔG) of the protein-ligand complex satisfies one or more thresholds, the ligand and the protein molecule may be determined to exhibit sufficient binding affinity. Contrastingly, where the binding free energy (ΔG) of the protein-ligand complex fails to satisfy the one or more thresholds, the ligand and the protein molecule may be determined to exhibit insufficient binding affinity.

FIG. 2B depicts a flowchart illustrating an example of a process 250 for determining the native pose (or ground-truth pose) of a protein molecule bound to a ligand, in accordance with some example embodiments. Referring to FIGS. 1 and 2B, the process 250 may be performed by the analysis controller 110, for example, by the docking engine 112, the simulation engine 114, and the evaluation engine 116. In some cases, the process 250 may be performed to determine the native pose (or ground-truth pose) of a protein molecule bound to a ligand. As noted, the native pose (or ground-truth pose) between a protein molecule and a ligand may be a docked pose that is most likely to be consistent with experimental observations (e.g., X-ray crystallography) of the pose assumed by the protein molecule and the ligand when the protein molecule is bound to the ligand. As described in more details below, one or more rounds of steered molecular dynamics (SMD) simulation may be performed to identify, as the native pose (or ground-truth pose) of the protein molecule bound to the ligand, a docked pose with the lowest binding free energy. However, instead of performing steered molecular dynamics (SMD) simulations on to determine the binding free energy of every possible docked pose between the protein molecule and the ligand, the process 250 may reduce the computational burden of the analysis by at least identifying a strategic subset of possible docked poses, such as those in which the ligand is oriented to bind to the protein molecule at more than a threshold portion of the binding site in the ligand and/or less than a threshold portion of the non-binding site in the ligand.

At 252, at least a portion of a binding site in a ligand forming an interface between the ligand and a protein molecule may be identified. In some example embodiments, the binding site of the ligand may include one or more portions of the ligand participating in a binding interaction with the protein molecule (e.g., by the analysis controller 110). Depending on the type of the ligand, the binding site may include one or more amino acid residues (where the ligand is another protein molecule), atoms (where the ligand is a small molecule), or nucleotides (where the ligand is a nucleic acid molecule) at an interface between the ligand and the protein molecule. Where the ligand is an antigen and the protein molecule is an antibody or a T-cell receptor (TCR), for example, the binding site of the ligand may be the epitope, which is recognized and bound to by the paratope (or the antigen-binding site) in the antibody or the T-cell receptor (TCR) during a binding interaction therebetween. In some cases, the binding site of the ligand may be determined based on experimental data indicating where the binding interaction between the ligand and protein molecule is more likely to occur, thus identifying, for example, the one or more of the amino acid residue, atoms, or nucleotides in the ligand participating in the binding interaction with the protein molecule. Examples of such experimental data include mass spectrometry cross-linking, alanine scanning, epitope mapping, and/or the like.

At 254, the analysis controller 110 may generate plurality of protein-ligand complexes by at least sampling a plurality of poses of the ligand in a binding site of the protein molecule. In some example embodiments, protein-ligand docking may include sampling a plurality of poses of the ligand bound to the protein molecule. However, some poses of the ligand, such as those in which the ligand is oriented to bind to the protein molecule at less than a threshold portion of the binding site in the ligand and/or more than a threshold portion of a non-binding site in the ligand, yield unstable protein-ligand complexes that should not undergo further native pose analysis. Accordingly, in some cases, the protein-ligand docking may be constrained by the binding site in the ligand such that the plurality of poses sampled excludes those poses in which the ligand is oriented to bind to the protein molecule at less than the threshold portion of the binding site and/or more than the threshold portion of the non-binding site in the ligand. The exclusion of these poses during protein-ligand docking may generate a subset of the possible protein-ligand complexes formed by the ligand bound to the protein molecule. In particular, the subset of protein-ligand complexes, which then undergo steered molecular dynamics (SMD) simulation to identify the native pose of the protein molecule bound to the ligand, may exclude unstable protein-ligand complexes in which the ligand is oriented to bind to the protein molecule at less than the threshold portion of the binding site and/or greater than the threshold portion of the non-binding site in the ligand. Instead, the possible protein-ligand complexes that are included in the subset of protein-ligand complexes are those stable protein-ligand complexes in which the ligand is oriented to bind to the protein molecule at greater than the threshold portion of the binding site and/or less than the threshold portion of the non-binding site in the ligand.

In some cases, the protein-ligand complexes that are included in the subset of protein-ligand complexes may further exclude those that are determined to be energetically and/or structurally unfavorable. For example, in some cases, the subset of possible protein-ligand complexes that undergo steered molecular dynamics (SMD) simulation may further exclude those protein-ligand complexes whose bound state energy, which may be estimated based on the energy of various atomic interactions present in each protein-ligand complex, fails to satisfy one or more thresholds. Alternatively and/or additionally, the subset of possible protein-ligand complexes may exclude one or more protein-ligand complexes based on shape complementarity and/or the quantity of polar interactions in the interface between the constituent protein molecule and ligand. Accordingly, in some cases, even when a protein-ligand complex is formed by the ligand oriented to bind to the protein molecule at greater than the threshold portion of the binding site and/or less than the threshold portion of the non-binding site in the ligand, the protein-ligand complex may nevertheless be excluded from subsequent steered molecular dynamics (SMD) simulation if the protein-ligand complex is energetically and/or structurally unfavorable.

At 256, a steered molecular dynamics (SMD) simulation may be performed for each protein-ligand complex of the plurality of protein ligand complexes to determine a binding free energy of each protein-ligand complex. In some example embodiments, the simulation engine 114 may perform, for each protein-ligand complex in the subset of protein-ligand complex (e.g., containing stable protein-ligand complexes and excluding unstable protein-ligand complexes), one or more rounds of steered molecular dynamics (SMD) simulation. For example, for each protein-ligand complex, a single round of steered molecular dynamics (SMD) simulation may include applying a force to pull the ligand away from the protein molecule in order to dissociate the protein-ligand complex, or separate the ligand from the protein molecule. As described in more detail below, the result of each round of steered molecular dynamics (SMD) simulation may include a quantity of work performed to dissociate the protein-ligand complex which, in some cases, corresponds to a binding free energy of the protein-ligand complex.

Where multiple rounds of steered molecular dynamics (SMD) simulation are performed for a single protein-ligand complex, each individual round of steered molecular dynamics (SMD) simulation may include subjecting the protein-ligand complex to identical external conditions such as, for example, a constant pulling force, a constant pulling velocity, constant pulling angles, and/or the like. Moreover, in some cases, one or more hyperparameters of the steered molecular dynamics (SMD) simulation may be adjusted to reduce or minimize variability in the results across multiple rounds of steered molecular dynamics (SMD) simulation performed for the same protein-ligand complex. For example, in some cases, the rate at which a protein-ligand complex is dissociated during each round of steered molecular dynamics (SMD) simulation may be reduced in order to reduced. Doing so may slow the transition of the protein-ligand complex from the bound state to the unbound state, thus reducing (or minimizing) the disparities between the quantity of work performed to dissociate the same protein-ligand complex across different rounds of steered molecular dynamics (SMD) simulation. Other examples of hyperparameters that may be adjusted (or tuned) include the presence or absence of solvent molecules surrounding the protein-ligand complex, the duration that the protein-ligand complex is pulled to dissociate the protein-ligand complex, the quantity of rounds of steered molecular dynamics (SMD) simulation performed for the protein-ligand complex, and/or the like.

At 258, a protein-ligand complex from the plurality of protein-ligand complex is identified as exhibiting a ground-truth pose of the ligand docked in the binding site of the protein molecule based at least on the binding free energy of each protein-ligand complex. In some example embodiments, for each protein-ligand complex in the subset of protein-ligand complexes generated through binding site constrained protein-ligand docking, the quantity of work that is performed to dissociate the protein-ligand complex across one or more rounds of steered molecular dynamics (SMD) simulation. As noted, the quantity of work performed to dissociate a protein-ligand complex may correspond to the binding free energy of the protein-ligand complex. In instances where multiple rounds of steered molecular dynamics (SMD) simulation are performed for a single protein-ligand complex, the quantity of work performed to dissociate the protein-ligand complex may correspond to a mean (or average), median, mode, maximum, minimum, or range of the quantity of work performed to dissociate the protein-ligand complex across the multiple rounds of steered molecular dynamics (SMD) simulation.

In some cases, upon performing the multiple rounds of steered molecular dynamics (SMD) simulation for each protein-ligand complex in the subset of protein-ligand complexes, each protein-ligand complex in the subset of protein-ligand complexes may be associated with a binding free energy corresponding to the quantity of work performed to dissociate the protein-ligand complex. In some cases, a protein-ligand complex with a lower binding free energy is considered more stable than another protein-ligand complex with a higher binding free energy. As such, where the binding free energy of a protein-ligand complex satisfy one or more criteria, that protein-ligand complex may be identified as having a ground-truth pose (or native pose) of the ligand docked in the binding site of the protein molecule. In some cases, the one or more criteria may include the binding free energy of the protein-ligand complex satisfying one or more threshold values. Alternatively and/or additionally, the one or more criteria may include the protein-ligand complex having the lowest binding energy (or one of the lowest binding free energies) in the subset of protein-ligand complexes.

To further illustrate, consider a first protein-ligand complex and a second protein-ligand complex from the subset of protein-ligand complexes. For the first protein-ligand complex in the subset of protein-ligand complexes generated through binding site constrained protein-ligand docking, multiple rounds of steered molecular dynamics (SMD) simulation may be performed. Each round of steered molecular dynamics (SMD) simulation may include applying a force to dissociate the first protein-ligand complex. Furthermore, the quantity of work required to dissociate the first protein-ligand complex may correspond to a mean (or average), median, mode, maximum, minimum, or range of the results from the multiple rounds of steered molecular dynamics (SMD) simulation. Similarly, multiple rounds of steered molecular dynamics (SMD) simulation may also be performed to dissociate the second protein-ligand complex. The quantity of work required to dissociate the second protein-ligand complex may correspond to a mean (or average), median, mode, maximum, minimum, or range of the results from these rounds of steered molecular dynamics (SMD) simulation. As described in more detail below, the first protein-ligand complex or the second protein-ligand complex may be identified as corresponding to the ground-truth pose (or native pose) of the ligand bound to the protein molecule based at least on the respective quantity of work required to dissociate each protein-ligand complex.

In some cases, the quantity of work performed to dissociate a protein-ligand complex may correspond to the degree of correspondence between the protein-ligand complex and the ground-truth pose (or native pose) of the protein molecule bound to the ligand. Thus, that the quantity of work performed to dissociate the first protein-ligand complex is greater than the quantity of work performed to dissociate the second protein-ligand complex may be an indication that, the first protein-ligand complex exhibits a greater degree of correspondence to the ground-truth pose (or native pose) of the protein molecule bound to the ligand than the second protein-ligand complex. Moreover, in some cases, the first protein-ligand complex may be identified as corresponding to the ground-truth pose of the ligand in the binding site of the protein molecule if the binding free energy of the first protein-ligand complex, which corresponds to the quantity of work performed to dissociate the first protein-ligand complex, satisfies one or more threshold values. Alternatively and/or additionally, the first protein-ligand complex may be identified as corresponding to the ground-truth pose of the ligand in the binding site of the protein molecule if the binding free energy of the first protein-ligand complex is lower than that of the other protein ligand complexes including, for example, the second protein-ligand complex. For instance, in some cases, the first protein-ligand complex may be identified as corresponding to the ground-truth pose of the ligand in the binding site of the protein molecule if the first protein-ligand complex has the lowest binding free energy of the protein-ligand complexes in the subset of protein-ligand complexes.

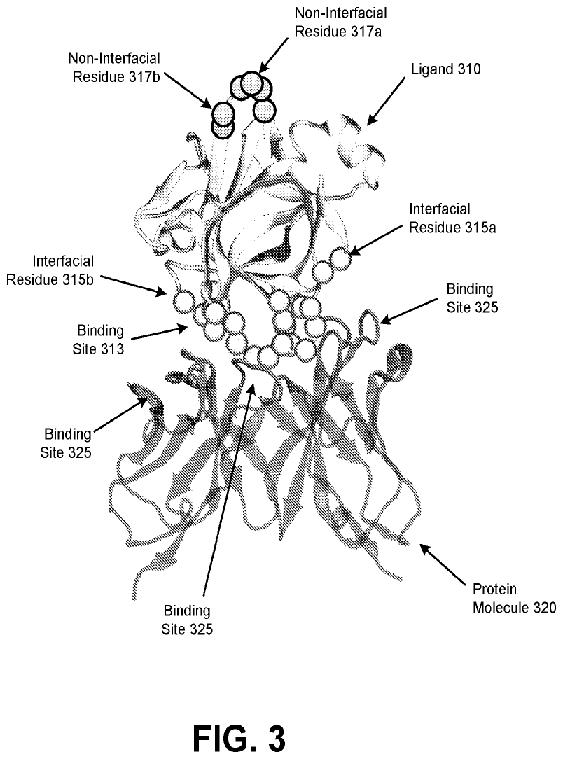

FIG. 3 depicts an example of protein-ligand docking between a ligand 310 and a protein molecule 320, in accordance with some example embodiments. In some example embodiments, the docking engine 112 may identify, in a binding site 313 in the ligand 310. In the example shown in FIG. 3, the ligand 310 is another protein molecule having a sequence of amino acid residues. Accordingly, as shown in FIG. 3, the binding site 313 in the ligand 310 may include a set of interfacial residues including, for example, a first interfacial residue 315a, a second interfacial residue 315b, and/or the like. Moreover, while the docking engine 112 performs protein-ligand docking by at least sampling different poses of the ligand 310 in a binding site 325 in the protein molecule 320, the docking engine 112 may impose one or more constraints based at least on the binding site 313 (e.g., the set of interfacial residues) in the ligand 310 such that the poses sampled by the docking engine 112 excludes poses in which the ligand 310 is oriented to bind to the protein molecule 320 at fewer than a threshold quantity of interfacial residues and/or greater than a threshold quantity of non-interfacial residues. The resulting set of protein-ligand complexes formed by the protein molecule 320 and the ligand 310 may include, for example, one or more protein-ligand complexes in which the ligand 310 is oriented to bind to the protein molecule 320 at the binding site 313 in the ligand 310 including, for example, the first interfacial residue 315a, the second interfacial residue 315b, and/or the like. However, the set of protein-ligand complexes may exclude one or more protein-ligand complexes in which the ligand 310 is oriented to bind to the protein molecule at a non-binding site in the ligand 310, which may include one or more non-interfacial residues such as, for example, the first non-interfacial residue 317a, the second non-interfacial residue 317b, and/or the like.

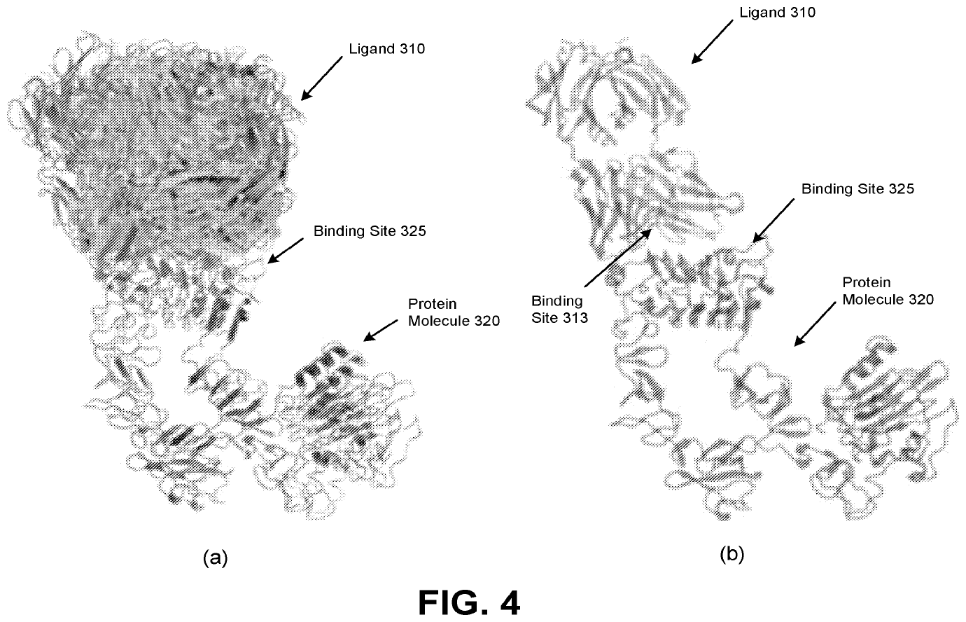

The practical effects of constraining the docking of the ligand 310 based on the binding site 313 in the ligand 310 (e.g., including the set of interfacial residues containing the first interfacial residue 315a, the second interfacial residue 315b, and/or the like) are shown in FIG. 4. Panel (a) on the left of FIG. 4 shows the numerous poses the ligand 310 can assume in the binding site 325 of the protein molecule 320 when protein-ligand docking is performed without any constraints. Evaluating each possible pose of the ligand 310 during protein-ligand docking is computationally inefficient at least because, as noted, the poses in which the ligand 310 is oriented to bind to the protein molecule 320 at non-interfacial residues (e.g., the first non-interfacial residue 317a, the second non-interfacial residue 317b, and/or the like) are immaterial given the improbability of these non-interfacial residues interacting with the binding site 325 of the protein molecule 320 to form a stable protein-ligand complex. Accordingly, in the example of protein-ligand docking shown in panel (b) on the right of FIG. 4, constraining protein-ligand docking to the binding site 313 in the ligand 310 to avoid poses in which the ligand 310 is oriented to bind to the protein molecule 320 at non-interfacial residues may reduce the quantity of poses that are sampled during protein-ligand docking. In particular, the poses that are excluded during protein-ligand docking are those that are unlikely to give rise to stable protein-ligand complexes. Accordingly, the imposition of the binding site based constraints may reduce the computation burden associated with protein-ligand docking and downstream binding affinity and native pose analysis without diminishing the accuracy of the results.

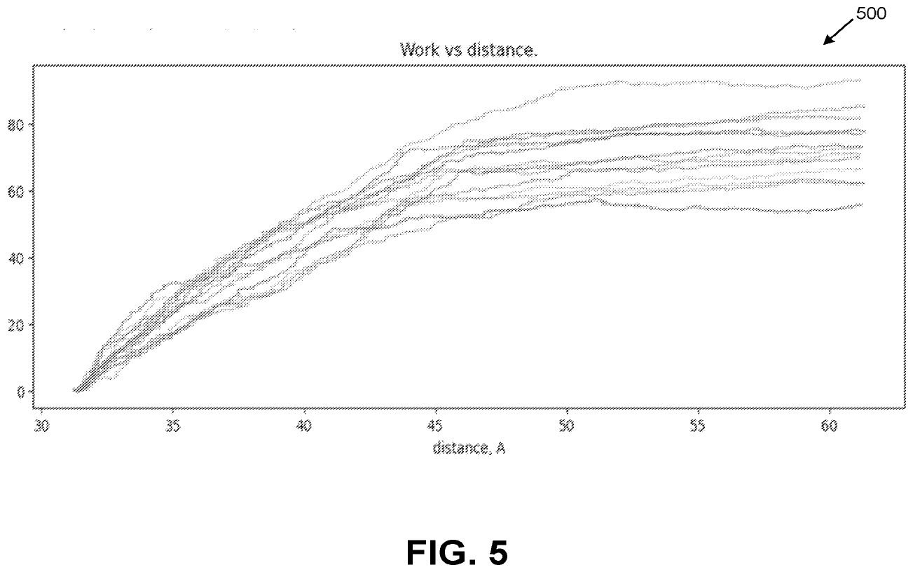

FIG. 5 depicts a graph 500 illustrating the work versus separation distance curves of multiple rounds of steered molecular dynamics simulation to dissociate a protein-ligand complex. Despite being exposed to identical external conditions, the quantity of work performed to dissociate the protein-ligand complex during a first round of steered molecular dynamics (SMD) simulation may differ from the quantity of work performed to dissociate the protein-ligand complex during a second round of steered molecular dynamics (SMD) simulation. Accordingly, as shown in Equation (1) above, the binding free energy of the protein-ligand complex corresponds to the mean (or average), median, mode, maximum, minimum, or range of the quantity of work performed to dissociate the protein-ligand complex across multiple rounds of steered molecular dynamics (SMD) simulation. Moreover, in some cases, the variation across different rounds of steered molecular dynamics (SMD) simulation may be reduced or minimized by adjusting one or more hyperparameters of the steered molecular dynamics (SMD) simulation. For example, variability in the outcome of different rounds of steered molecular dynamics (SMD) simulation on the same protein-ligand complex may be reduced by protracting each round of steered molecular dynamics (SMD) simulation such that the protein-ligand complex is able to reach closer to thermodynamic equilibrium during the states subsequent to its initial equilibrium state as a bound molecule.

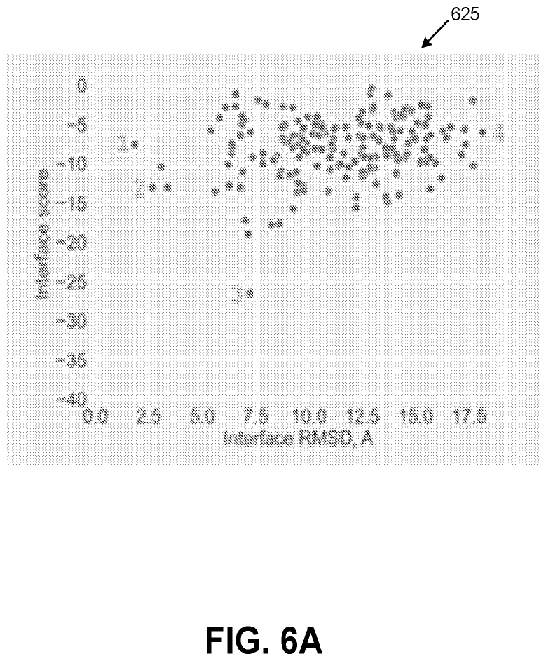

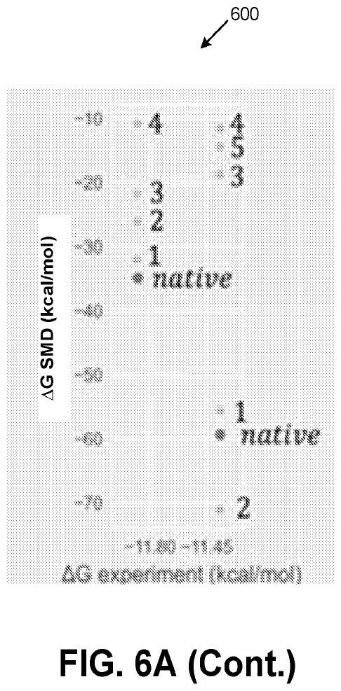

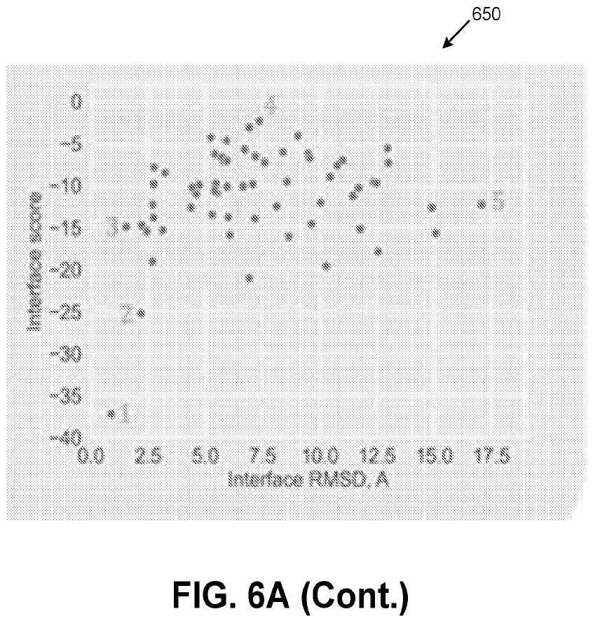

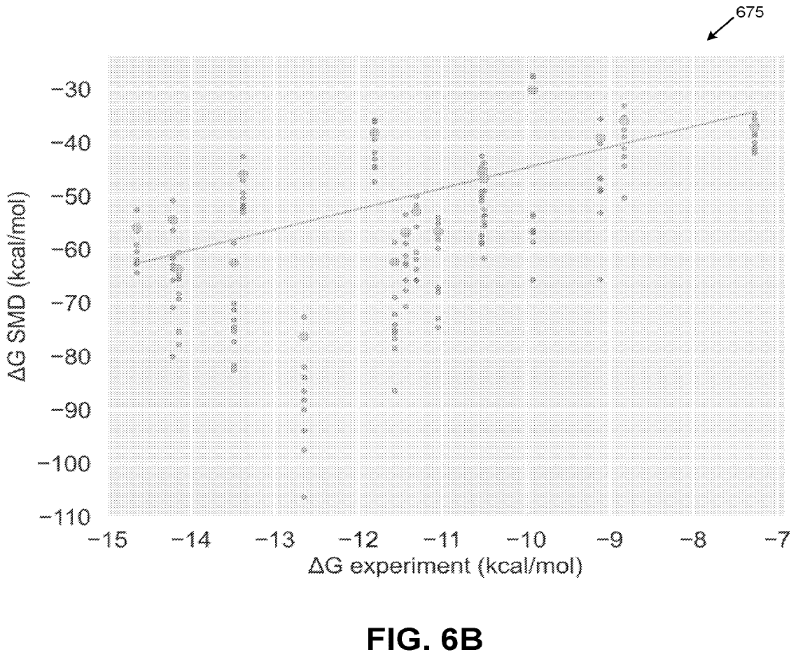

FIG. 6A depicts various graphs illustrating the application of steered molecular dynamics (SMD) simulation in identifying the native pose (or ground-truth pose) of a protein molecule bound to a ligand, in accordance with some example embodiments. As shown in graph 600 in FIG. 6A (center panel), the level of correspondence between a protein-ligand complex and the native pose (or ground-truth pose) of the constituent protein molecule bound to the ligand may be determined based on the binding free energy (ΔG) of the protein-ligand complex which, as noted, corresponds to the quantity of work performed to dissociate the protein-ligand complex during one or more rounds of steered molecular dynamics (SMD) simulation. Graph 600 shows the spread in binding free energy (ΔG) between two different sets of protein-ligand complexes (including points labeled 1 through 4 and 1 through 5) and the corresponding native pose (or ground-truth pose). The native pose (or ground-truth pose) of a protein molecule bound to a ligand may be associated with a lower binding free energy (ΔG) than the non-native poses (or non-ground truth poses) of the protein molecule bound to the ligand. As noted, the binding free energy (ΔG) of a protein-ligand complex may correspond the difference between a first energy of the protein-ligand complex in a bound state and a second energy of the protein-ligand complex in an unbound state. A lower binding free energy (ΔG) may correspond to a greater reduction in the energy when a protein molecule and a ligand are in a bound state, thus indicating greater stability in the corresponding protein-ligand complex. Accordingly, the binding free energy (ΔG) of a protein-ligand complex may be lower for those protein-ligand complexes that bear a closer resemblance to the native pose (or ground-truth pose).

The nexus between the binding free energy (ΔG) of a protein-ligand complex and its correspondence to the native pose (or ground-truth pose) of the constituent protein molecule and ligand is shown in graph 600. For example, in graph 600, the points labeled “1” and “2” in the respective set of protein-ligand complexes may be identified as exhibiting the highest degree of correspondence to the native pose (or ground-truth pose) of the corresponding protein molecule and ligand pair based at least on these points being associated with the lowest binding free energy (ΔG). It should be appreciated that binding free energy (ΔG) provides a more accurate metric for native pose (or ground-truth pose) identification than the interface score used in conventional techniques. In particular, graphs 625 and 650 show that the protein-ligand complexes that are correctly identified, based on binding free energy (ΔG), as being closer matches to the respective native poses (or ground-truth poses) are not correctly identified as such based on the interface score of conventional techniques. For instance, in graphs 625 and 650, the difference between a protein-ligand complex and the corresponding native pose (or ground-truth pose) is quantified based on interface root mean square distance (RMSD). However, in graphs 625 and 650, those protein-ligand complexes (e.g., point “3” in graph 625) whose high interface root mean square distance (RMSD) indicates a large discrepancy relative to the corresponding native pose (or ground-truth pose) are assigned low interface scores, meaning that conventional interface scores would incorrectly identify these protein-ligand complexes as corresponding to the native pose (or ground-truth pose) of the constituent protein molecule and ligand. Meanwhile, those protein-ligand complexes (e.g., points “1” and “2” in graph 625) whose low interface root mean square distance (RMSD) indicates a closer match to the corresponding native pose (or ground-truth pose) are assigned high interface scores, meaning that conventional interface scores would fail to correctly identify these protein-ligand complexes as corresponding to the native pose (or ground-truth pose) of the constituent protein molecule and ligand. Contrastingly, as shown in graph 600, the binding free energy (ΔG) of each protein-ligand complex is consistent with the degree of correspondence to the corresponding native pose (or ground-truth pose). In particular, those protein-ligand complexes (e.g., point “4” in graph 625 and point “5” in graph 650) that fail to match the corresponding native pose (or ground-truth pose), as indicated by their high root mean square distance (RMSD), are shown in graph 500 as being associated with high binding free energies (ΔG).

FIG. 6B depicts a graph 675 illustrating the relationship between the binding free energies (ΔG) determined by performing steered molecular dynamics (SMD) simulations on protein-ligand complexes identified through experimentation and the corresponding experimentally derived values. In the graph 675, the experimentally derived binding free energies (ΔG) are plotted against the x-axis while the binding free energies (ΔG) derived through steered molecular dynamics (SMD) simulations are plotted against the y-axis. The smaller dots represent the binding free energies (ΔG) determined through steered molecular dynamics (SMD) simulations. Each vertical column of smaller dots correspond to the range of binding free energies (ΔG) determined for a single protein-ligand complex across multiple rounds of steered molecular dynamics (SMD) simulations. The large dot in each column represents the mean (or average) binding free energy (ΔG) for that group of binding free energy values. The line plotted across represent the experimental values of binding free energies (ΔG). For these steered molecular dynamics (SMD) simulations, each protein-ligand complex was subjected to a pulling velocity of 1 angstrom per nanosecond. The coefficient of determination (R²) for the steered molecular dynamics (SMD) simulations was 0.42. The results in FIG. 6B evidence the correlation between the binding affinity computed through steered molecular dynamics (SMD) simulations and the “true” binding affinity determined through experimentation.

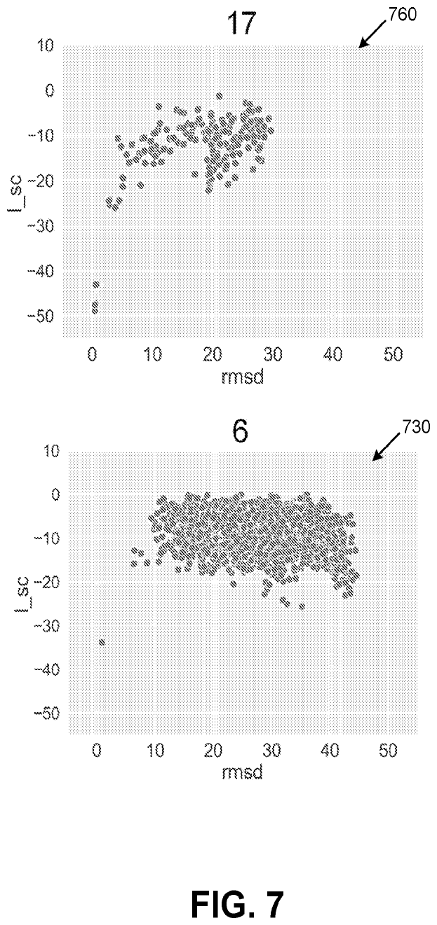

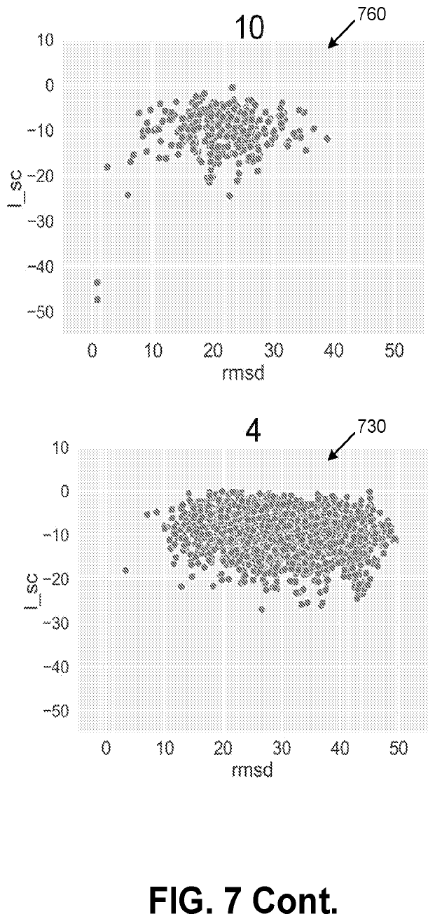

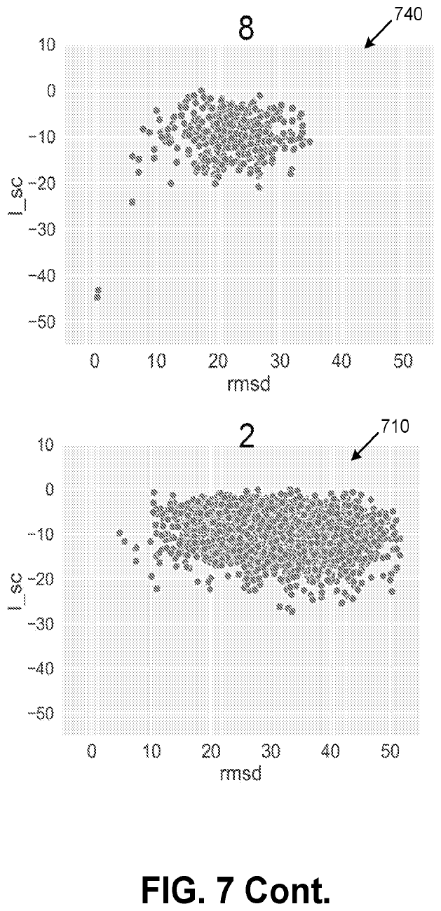

As noted, the protein-ligand docking performed by the docking engine 112, for example, in operation 204 of the process 200 in FIG. 2A and operation 254 of the process 250 in FIG. 2B, may be constrained based on a threshold portion of the binding site in the ligand. In some cases, the threshold portion of the binding site in the ligand may include, depending on the type of ligand, a threshold quantity of the amino acid residues, atoms, or nucleotides forming the interface between the ligand and the protein molecule. Moreover, in some cases, the threshold quantity of amino acid residues, atoms, or nucleotides may include a maximum quantity and/or a minimum quantity. FIG. 7 depicts graphs illustrating the effects of constraining protein-ligand docking to different quantities of interfacial amino acid residues, in accordance with some example embodiments. Referring to FIG. 7, the effects of constraining protein-ligand docking to 2 amino acid residues (graph 710), 4 amino acid residues (graph 720), 6 amino acid residues (graph 730), 8 amino acid residues (graph 740), 10 amino acid residues (graph 750), and 17 amino acid resides (graph 760) are shown in graphs plotting the relationship between the interface score indicative of the computationally predicted quality of the binding interface between a ligand and a protein molecule and the interface root mean square distance (RMSD) indicative of the measured quality of the binding interface between the ligand and the protein molecule.

In this context, interface root mean square distance (RMSD) may measure the difference between the binding interface determined through protein-ligand docking and the actual binding interface (or experimentally observed binding interface) of the protein-ligand complex in a native pose (or ground-truth pose) in which the ligand is docked in a binding site of the protein molecule. Thus, a protein-ligand complex exhibiting a lower root mean square distance (RMSD) may be a closer match to the native pose (or ground-truth pose) of the ligand docked in the binding site of the protein molecule than another protein-ligand complex having a higher root mean square distance (RMSD). It should be appreciated that protein-ligand docking may be constrained based on a minimum quantity of interfacial residues (e.g., two interfacial residues and/or the like) in order to achieve meaningful reduction in computational burden. Moreover, FIG. 7 shows that as the docking engine 112 constrains protein-ligand docking based on a larger quantity of interfacial residues, more of the protein-ligand complexes identified as stable through protein-ligand docking (as indicated by lower interface scores) may also exhibit a low interface root mean square distance (RMSD) meaning that these protein-ligand complexes have a binding interface that match (or nearly match) the corresponding ground truth binding interface. In other words, in addition to reducing the computational burden associated with protein-ligand docking, constraining protein-ligand docking based on more amino acid residues may also increase the accuracy of the predictions made by docking engine 112. That is, more constraints may increase the likelihood of achieving protein-ligand complexes with a low root mean square distance (RMSD) relative to the native pose (or ground-truth pose) of the ligand docked in the binding site of the protein molecule. However, acquiring more constraints, which includes identifying more interfacial residues, imposes higher upfront cost as experimentation (e.g., alanine scanning, crosslinking, and/or the like) are typically necessary to identify such constraints.

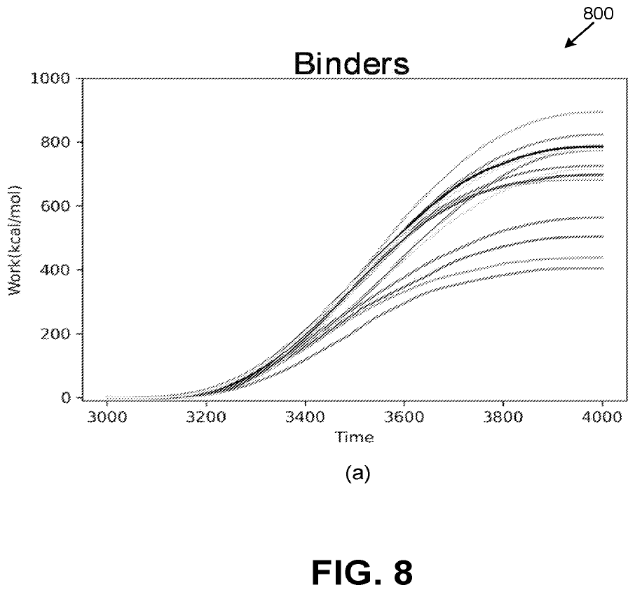

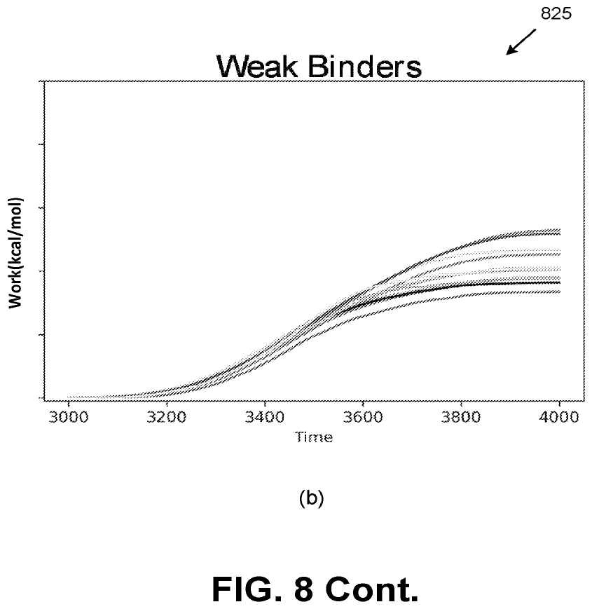

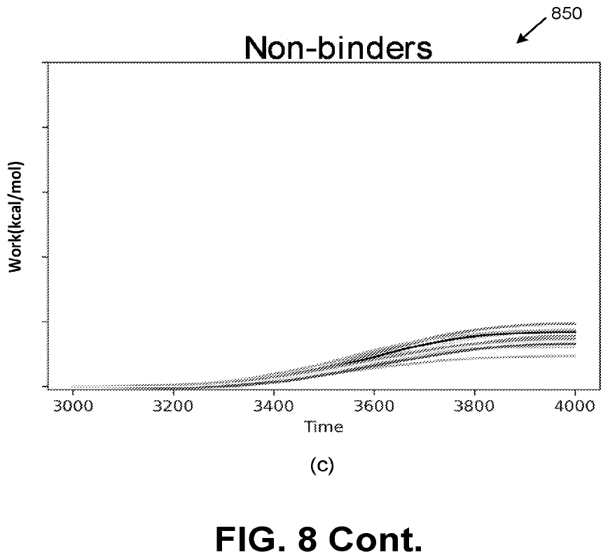

FIG. 8 depicts graphs illustrating the relationship between the binding affinity between a ligand and a protein molecule and the work performed to dissociate a corresponding protein-ligand complex. In particular, graph 800 in panel (a) of FIG. 8 shows the work versus time graph of binders (or pairs of protein molecules and ligands exhibiting high binding affinity), graph 825 in panel (b) shows the work versus time graph of weak binders (pairs of protein molecules and ligands exhibiting low binding affinity), and graph 850 in panel (c) shows the work versus time graph of non-binders (or pairs of protein molecules and ligands exhibiting no binding affinity). The work versus time graphs shown in FIG. 8 are consistent with the relationship expressed in Equation (1) above in which the quantity of work performed to dissociate a protein-ligand complex is proportional to the binding free energy (ΔG) of the protein-ligand molecule. More work may be performed to dissociate a protein-ligand complex formed between two binders (or a protein molecule and a ligand pair exhibiting a high binding affinity). As such, in some example embodiments, the evaluation engine 116 may determine, based at least on the binding free energy (ΔG) of each protein-ligand complex (or the corresponding work performed to dissociate each protein-ligand complex), the binding affinity between the ligand and the protein molecule forming each protein-ligand complex.

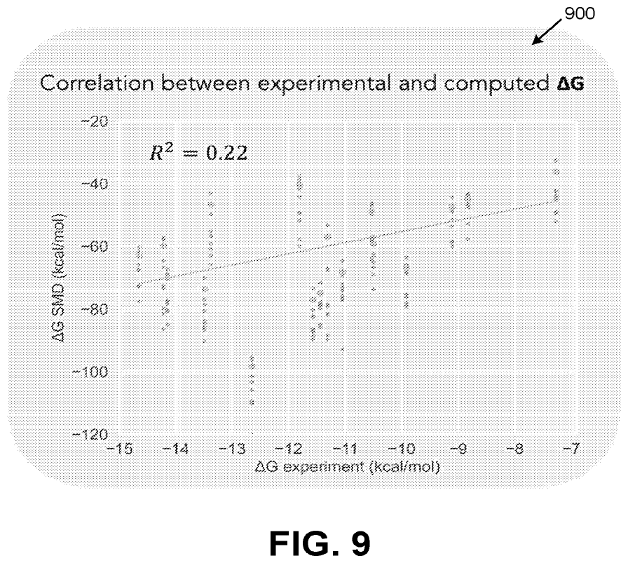

FIG. 9 depicts a graph 900 illustrating a comparison between experimentally derived binding free energies (ΔG) and computationally derived binding free energies (ΔG), in accordance with some example embodiments. In the example shown in FIG. 9, the computationally derived binding energies (ΔG) may refer to those binding energies determined by performing one or more rounds of steered molecular dynamics (SMD) simulation on a protein-ligand complex, such as a protein-ligand complex generated through protein-ligand docking constrained based on one or more interfacial residues of the ligand. For each protein-ligand complex, graph 900 includes a cluster of smaller dots corresponding to the computationally derived binding free energies (ΔG) (e.g., each small dot indicating the binding free energy determined by a single round of steered molecular dynamics (SMD) simulation) and a larger dot corresponding to the experimentally derived binding free energy (ΔG). Accordingly, graph 900 shows a correlation between the experimentally derived binding free energy (ΔG) for each protein-ligand complex and the corresponding computationally derived binding free energy (ΔG).

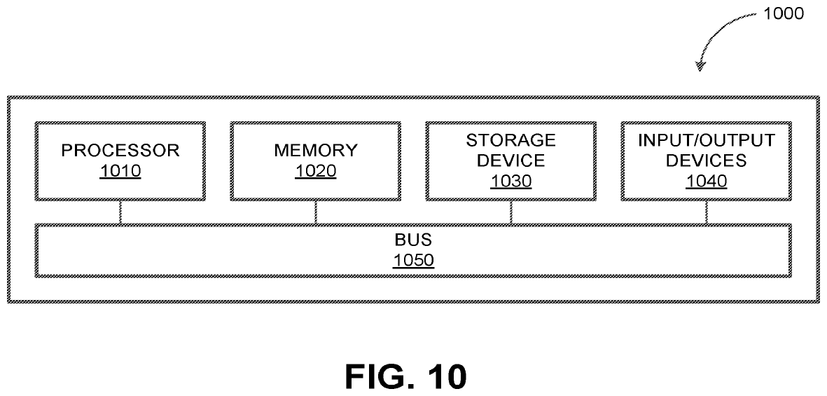

FIG. 10 depicts a block diagram illustrating an example of a computing system 1000, in accordance with some example embodiments. In some example embodiments, the computing system 1000 can be used to implement the analysis controller 110 and/or any components therein. As shown in FIG. 10, the computing system 1000 can include a processor 1010, a memory 1020, a storage device 1030, and input/output devices 1040. The processor 1010, the memory 1020, the storage device 1030, and the input/output devices 1040 can be interconnected via a system bus 1050. The processor 1010 is capable of processing instructions for execution within the computing system. Such executed instructions can implement one or more components of, for example, analysis controller 110 (e.g., Molecular dynamics simulation engine 114, Perturbation set engine 112, etc.). In some example embodiments, the processor 1010 can be a single-threaded processor. Alternately, the processor 1010 can be a multi-threaded processor. The processor 1010 is capable of processing instructions stored in the memory 1020 and/or on the storage device 1030 to display graphical information for a user interface provided via the input/output device 1040.

The memory 1020 is a computer readable medium such as volatile or non-volatile that stores information within the computing system 1000. The memory 1020 can store data structures representing configuration object databases, for example. The storage device 1030 is capable of providing persistent storage for the computing system 1000. The storage device 1030 can be a floppy disk device, a hard disk device, an optical disk device, a tape device, a solid-state drive, and/or other suitable persistent storage means. The input/output device 1040 provides input/output operations for the computing system 1000. In some example embodiments, the input/output device 1040 includes a keyboard and/or pointing device. In various implementations, the input/output device 1040 includes a display unit for displaying graphical user interfaces.

### DEFINITIONS

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

As used herein, the conformation of a protein molecule (or a protein conformation) is a three-dimensional arrangement of residues in a sequence of amino acid residues forming the protein molecule. A residue is an organic molecule that includes an alpha carbon linked to an amino group, a carboxyl group, a hydrogen atom, and a variable component called a side chain. As used herein, a protein-ligand complex is a structure formed as a result of intermolecular interactions (e.g., electrostatics, van der Waals forces, and/or the like) between a protein molecule (having a particular conformation) and a ligand. In some cases, the protein molecule and the compound in the coupled protein-structure-compound can be bound to one another (e.g., distance between the center of mass of the compound and the center of mass of the nitrogen atoms in the perturbation set of the protein molecule is smaller than a threshold distance value). In some cases, the protein molecule and the compound in the coupled protein-structure-compound are dissociated or not bound to one another (e.g., distance between the center of mass of the compound and the center of mass of the nitrogen atoms in the perturbation set of the protein molecule is greater than the threshold distance value). As used herein, structural information associated with a particular conformation of a protein molecule includes the spatial arrangement and/or orientation of atoms in each constituent amino acid residue forming the protein molecule (e.g., relative spatial locations and orientation of atoms in the protein molecule).

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B”.

It is understood that aspects and embodiments of the disclosure described herein include “comprising”, “consisting”, and “consisting essentially of” aspects and embodiments.

As used herein, “comprising” is synonymous with “including”, “containing”, or “characterized by”, and is inclusive or open-ended and does not exclude additional, unrecited elements or method steps. Any recitation herein of the term “comprising”, particularly in a description of components of a composition or in a description of steps of a method, is understood to encompass those compositions and methods consisting essentially of and consisting of the recited components or steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method.

Where a range of values is provided, it is understood by one having ordinary skill in the art that all ranges disclosed herein encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to”, “at least”, “greater than”, “less than”, and the like include the number recited and refer to ranges which can be subsequently broken down into sub-ranges as dis-cussed above. As will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value.

Headings, e.g., (a), (b), (i) etc., are presented merely for ease of reading the specification and claims. The use of headings in the specification or claims does not require the steps or elements be performed in alphabetical or numerical order or the order in which they are presented.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

Non-transitory computer program products (i.e., physically embodied computer program products) are also described that store instructions, which when executed by one or more data processors of one or more computing systems, causes at least one data processor to perform operations herein. Similarly, computer systems are also described that may include one or more data processors and memory coupled to the one or more data processors. The memory may temporarily or permanently store instructions that cause at least one processor to perform one or more of the operations described herein. In addition, methods can be implemented by one or more data processors either within a single computing system or distributed among two or more computing systems. Such computing systems can be connected and can exchange data and/or commands or other instructions or the like via one or more connections, including a connection over a network (e.g. the Internet, a wireless wide area network, a local area network, a wide area network, a wired network, or the like), via a direct connection between one or more of the multiple computing systems, etc.

The subject matter described herein may be embodied in systems, apparatus, methods, and/or articles depending on the desired configuration. For example, apparatuses and/or processes described herein can be implemented using one or more of the following: a processor executing program code, an application-specific integrated circuit (ASIC), a digital signal processor (DSP), an embedded processor, a field programmable gate array (FPGA), and/or combinations thereof. These various implementations may include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device. These computer programs (also known as programs, software, software applications, applications, components, program code, or code) include machine instructions for a programmable processor and may be implemented in a high-level procedural and/or object-oriented programming language, and/or in assembly/machine language. As used herein, the term “machine-readable medium” refers to any computer program product, computer-readable medium, computer-readable storage medium, apparatus and/or device (e.g., magnetic discs, optical disks, memory, Programmable Logic Devices (PLDs)) used to provide machine instructions and/or data to a programmable processor, including a machine-readable medium that receives machine instructions. Similarly, systems are also described herein that may include a processor and a memory coupled to the processor. The memory may include one or more programs that cause the processor to perform one or more of the operations described herein.

Although a few variations have been described in detail above, other modifications or additions are possible. In particular, further features and/or variations may be provided in addition to those set forth herein. Moreover, the implementations described above may be directed to various combinations and subcombinations of the disclosed features and/or combinations and subcombinations of several further features disclosed above. In addition, the logic flow depicted in the accompanying figures and/or described herein does not require the particular order shown, or sequential order, to achieve desirable results. Other implementations may be within the scope of the following claims. Furthermore, the specific values provided in the foregoing are merely examples and may vary in some implementations.

Although various aspects of the present disclosure are set out in the claims, other aspects of the present disclosure comprise other combinations of features from the described implementations with the features of the claims, and not solely the combinations explicitly set out in the claims.

1. A system, comprising:
at least one data processor; and
at least one memory storing instructions, which when executed by the at least one data processor, result in operations comprising:
identifying at least a portion of a binding site in a ligand forming an interface between the ligand and a protein molecule;
generating one or more protein-ligand complexes by at least sampling a plurality of different poses of the ligand in a binding site in the protein molecule,
the plurality of different poses being constrained by the binding site of the ligand such that each pose of the plurality of poses orients the binding site of the ligand to bind to the binding site of the protein molecule;
performing, for each protein-ligand complex of the one or more protein-ligand complexes, a steered molecular dynamics (SMD) simulation to determine a binding free energy of each protein-ligand complex; and
determining, based at least on the binding free energy of each protein-ligand complex, a binding affinity between the ligand and the protein molecule.

2. The system of claim 1, wherein the ligand is another protein molecule comprising a sequence of amino acid residues, and wherein the binding site of the ligand comprises a set of interfacial residues comprising one or more amino acid residues forming at least a portion of the interface between the ligand and the protein molecule.

3. The system of claim 2, further comprising:
selecting, from a second plurality of amino acid residues forming the interface between the ligand and the protein molecule, one or more amino acid residues for inclusion in the set of interfacial residues.

4. The system of claim 3, wherein a threshold quantity of amino acid residues from the second plurality of amino acid residues are selected for inclusion in the set of interfacial residues.

5. The system of claim 3, wherein the one or more amino acid residues are selected randomly from the second plurality of amino acid residues.

6. The system of claim 3, wherein the one or more amino acid residues are selected based on at least one of (i) a location of the one or more amino acid residues in the binding site of the ligand (ii) a location of the one or more amino acid residues in the ligand as a whole, (iii) a likelihood of the one or more amino acid residue participating in a binding interaction with the protein molecule, and (iv) a type of the one or more amino acid residues.

7. The system of claim 2, wherein the set of interfacial residues is identified based at least on experimental data identifying one or more amino acid residues participating in a binding interaction between the ligand and the protein molecule.

8. The system of claim 1, wherein the ligand is an antigen and the protein molecule is an antibody or a T-cell receptor (TCR), and wherein the binding site of the ligand includes an epitope in the antigen recognized by the antibody or the T-cell receptor (TCR).

9. The system of claim 1, wherein the ligand is an enzyme inhibitor and the protein molecule is an enzyme.

10. The system of claim 1, wherein the ligand is a small molecule comprising a plurality of atoms, and wherein the binding site of the ligand includes one or more atoms in the small molecule participating in a binding interaction with the protein molecule.

11. The system of claim 1, wherein the ligand is a nucleic acid molecule and the binding site of the ligand includes one or more nucleotides in the nucleic acid molecule participating in a binding interaction with the protein molecule.

12. The system of claim 1, wherein an individual round of steered molecular dynamics (SMD) simulation includes dissociating the protein-ligand complex by at least pulling the ligand away from the protein molecule with a constant force and/or a constant velocity over a period of time.

13. (canceled)

14. The system of claim 1, further comprising:
performing, for each protein-ligand complex, a plurality of rounds of steered molecular dynamics (SMD) simulation;
determining, for each round of steered molecular dynamics (SMD) simulation, a quantity of work performed to dissociate the protein-ligand complex, wherein the quantity of work performed to dissociate the protein-ligand complex in each round of steered molecular dynamics (SMD) simulation corresponds to a quantity of work performed to transition the protein-ligand complex from a bound state to an unbound state; and
determining, based at least on the quantity of work performed for each round of steered molecular dynamics (SMD) simulation, the binding free energy of each protein-ligand complex of the one or more protein-ligand complexes, wherein the binding free energy of each protein-ligand complex corresponds to at least one of (i) an average of the quantity of work performed across the plurality of rounds of steered molecular dynamics (SMD) simulation and (ii) a difference between an energy of the protein-ligand complex in the bound state and an energy of the protein-ligand complex in the unbound state.

15. (canceled)

16. (canceled)

17. (canceled)

18. The system of claim 1, further comprising:
adjusting one or more hyperparameters of the steered molecular dynamics (SMD) simulation to reduce a difference between the binding free energy of a same protein-ligand complex across different rounds of steered molecular dynamics (SMD) simulation, wherein the one or more hyperparameters include at least one of (i) a presence or absence of solvent molecules surrounding each protein-ligand complex, (ii) a rate at which each protein-ligand complex is pulled to dissociate the protein-ligand complex, (iii) a duration that each protein-ligand complex is pulled to dissociate each protein-ligand complex, and (iv) a quantity of rounds of steered molecular dynamics (SMD) simulation performed for each protein-ligand complex.

19. (canceled)

20. (canceled)

21. The system of claim 1, further comprising:
determining a plurality of protein-ligand complexes by at least sampling the plurality of different poses of the ligand in the binding site of the protein molecule; and
selecting, from the plurality of protein-ligand complexes, the one or more protein-ligand complexes for performing the steered molecular dynamics (SMD) simulation, wherein the one or more protein-ligand complexes are selected based on at least one of (i) an energy of each protein-ligand complex of the plurality of protein-ligand complexes in a bound state; (ii) a shape complementarity between the ligand and the protein molecule in each protein-ligand complex, and (iii) a quantity of polar interactions across an interface between the ligand and the protein molecule in each protein-ligand complex.

22. (canceled)

23. The system of claim 1, wherein the generating the one or more protein-ligand complexes includes
docking, to the protein molecule, the ligand in a pose in which the ligand is oriented to bind to the protein molecule at least a threshold portion of the binding site of the ligand while excluding, from being docked to the protein molecule, the ligand in a different pose in which the ligand is oriented to (i) bind to the protein molecule at less than the threshold portion of the binding site in the ligand or (ii) bind to the protein molecule at greater than a threshold portion of a non-binding site in the ligand.

24. (canceled)

25. (canceled)

26. (canceled)

27. The system of claim 1, wherein the binding site of the protein molecule includes at least a portion of (i) one or more antigen binding fragments (Fab) of the protein molecule, (ii) a variable domain (Fv) of the protein molecule, or (iii) one or more complementarity-determining regions (CDRs) of the protein molecule.

28. The system of claim 1, wherein the binding site of the protein molecule is an active site where a substrate binds to the protein molecule or an allosteric site.

29. A computer-implemented method, comprising:
identifying at least a portion of a binding site in a ligand forming an interface between the ligand and a protein molecule;
generating one or more protein-ligand complexes by at least sampling a plurality of different poses of the ligand in a binding site in the protein molecule,
the plurality of different poses being constrained by the binding site of the ligand such that each pose of the plurality of poses orients the binding site of the ligand to bind to the binding site of the protein molecule;
performing, for each protein-ligand complex of the one or more protein-ligand complexes, a steered molecular dynamics (SMD) simulation to determine a binding free energy of each protein-ligand complex; and
determining, based at least on the binding free energy of each protein-ligand complex, a binding affinity between the ligand and the protein molecule.

30. A non-transitory computer readable medium storing instructions, which when executed by at least one data processor, result in operations comprising:
identifying at least a portion of a binding site in a ligand forming an interface between the ligand and a protein molecule;
generating one or more protein-ligand complexes by at least sampling a plurality of different poses of the ligand in a binding site in the protein molecule,
the plurality of different poses being constrained by the binding site of the ligand such that each pose of the plurality of poses orients the binding site of the ligand to bind to the binding site of the protein molecule;
performing, for each protein-ligand complex of the one or more protein-ligand complexes, a steered molecular dynamics (SMD) simulation to determine a binding free energy of each protein-ligand complex; and
determining, based at least on the binding free energy of each protein-ligand complex, a binding affinity between the ligand and the protein molecule.

31-45. (canceled)
